(19) United States
(12) Patent Application Publication
(10) Pub. No.: US 2026/0321398 A1
(43) Pub. Date: Oct. 15, 2026

(54) MEDICAL SUPPORT APPARATUS, MEDICAL SUPPORT SYSTEM, AND MEDICAL SUPPORT METHOD

(71) Applicant: OLYMPUS MEDICAL SYSTEMS CORP., Tokyo (JP)

(72) Inventors: Yuzuko NIIGAKI, Tokyo (JP); Yuta KOBAYASHI, Tokyo (JP); Tatsuki ODEISHI, Tokyo (JP); Hayato KOBAYASHI, Tokyo (JP)

(73) Assignee: OLYMPUS MEDICAL SYSTEMS CORP., Tokyo (JP)

(21) Appl. No.: 19/464,980

(22) Filed: Jan. 30, 2026

Related U.S. Application Data
(63) Continuation of application No. PCT/JP2023/028350, filed on Aug. 2, 2023.

(51) Int. Cl. G16H 30/40; G06T 7/00; G16H 30/20; G16H 50/20

(52) U.S. Cl. CPC G16H 30/40; G06T 7/0012; G16H 30/20; G16H 50/20; G06T 2207/10068; G06T 2207/30004

(57) ABSTRACT

A medical support apparatus includes: a processor configured to acquire a plurality of image files in which a plurality of images of an inside body of a subject captured at multiple imaging magnifications, imaging magnification information, and imaging times are associated, based on the imaging magnification information, set an image having a first imaging magnification among the plurality of images to a first image, set an image having a next first imaging magnification on in time series among the plurality of images to a second image, and determine images captured in a period from an imaging time of the first image to an imaging time of the second image as third images, group, based on a result of the determination result, the third images into an image group, select a representative image of the image group, and display the representative image on a display.

### CROSS REFERENCES TO RELATED APPLICATIONS

This application is a continuation of International Application No. PCT/JP2023/028350, filed on Aug. 2, 2023, the entire contents of which are incorporated herein by reference.

### BACKGROUND

### 1. Technical Field

The present disclosure is related to a medical support apparatus, a medical support system, and a medical support method for providing endoscopic medical support.

### 2. Related Art

A technology for displaying, in an endoscope, a plurality of endoscopic image groups for each lesion group is known (for example, see International Publication No. WO 2019/220801). In this technology, a group is formed for each of lesions by grouping endoscopic images by using a degree of correlation based on an image feature amount between lesion images that are detected from the endoscopic image groups, a representative image is selected in an endoscopic image group in the group, and the representative image and a lesion group are stored in an associated manner.

Further, a technology for displaying, in an endoscope, a desired number of endoscopic images when endoscopic images are acquired from an image storage server that manages endoscopic image groups is known (for example, see International Publication No. WO 2022/195696). In this technology, a simultaneously-captured image group that includes a plurality of endoscopic images for which at least one of an imaging method and image processing is different is acquired, the number of endoscopic images to be displayed from the simultaneously-captured image group is set, and the set number of endoscopic images are selected and displayed from the simultaneously-captured image group.

### SUMMARY

In some embodiments, a medical support apparatus includes: a processor comprising hardware, the processor being configured to acquire a plurality of image files in which a plurality of images of an inside body of a subject captured in temporal sequence at multiple imaging magnifications, imaging magnification information on the imaging magnifications, and imaging times of the plurality of images are associated, based on the imaging magnification information, set an image having a first imaging magnification among the plurality of images to a first image, set an image having a next first imaging magnification on in time series among the plurality of images to a second image, and determine images captured in a period from an imaging time of the first image to an imaging time of the second image as third images that include identical feature areas, group, based on a result of the determination result, the third images into an image group, select a representative image of the image group, and display the representative image on a display.

In some embodiments, a medical support system includes: an endoscope configured to capture an inside body of a subject; a controller configured to record a plurality of image files in which a plurality of images of the inside body of the subject captured in temporal sequence at multiple imaging magnifications by the endoscope, imaging magnification information on the imaging magnifications, and imaging times of the plurality of images are associated; and a medical support apparatus comprising a processor comprising hardware, the processor being configured to acquire the plurality of image files from the controller, based on the imaging magnification information, set an image having a first imaging magnification among the plurality of images to a first image, set an image having a next first imaging magnification on in time series among the plurality of images to a second image, and determine images captured in a period from an imaging time of the first image to an imaging time of the second image as third images that include identical feature areas, group, based on a result of the determination, the third images into an image group, select a representative image of the image group, and display the representative image on a display.

In some embodiments, a medical support method includes acquiring a plurality of image files in which a plurality of images of an inside body of a subject captured in temporal sequence at multiple imaging magnifications, imaging magnification information on the imaging magnifications, and an imaging times of the plurality of images are associated, based on the imaging magnification information, setting an image having a first imaging magnification among the plurality of images to a first image, setting an image having a next first imaging magnification on in time series among the plurality of images to a second image, and determining images captured in a period from an imaging time of the first image to an imaging time of the second image as third images that include identical feature areas, grouping, based on a result of the determination, the third images into an image group, selecting a representative image of the image group, and displaying the representative image on a display.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of a medical support system according to one embodiment;

FIG. 2 is a block diagram illustrating a functional configuration of main parts of an endoscope and a control apparatus according to one embodiment;

FIG. 3 is a block diagram illustrating a functional configuration of an image diagnosis apparatus according to one embodiment;

FIG. 4 is a block diagram illustrating a functional configuration of a medical support apparatus according to one embodiment;

FIG. 5 is a flowchart illustrating an outline of a process that is performed by the medical support apparatus according to one embodiment;

FIG. 6 is a diagram illustrating an example of contents of each of image files;

FIG. 7 is a diagram illustrating an example of region information that is included in examination result information;

FIG. 8 is a diagram illustrating an example of lesion size information that is about a feature area and that is included in the examination result information;

FIG. 9 is a diagram illustrating an example of treatment information that is included in the examination result information;

FIG. 10 is a diagram for explaining an outline of a determination method for determination performed by a determining unit that is included in the medical support apparatus according to one embodiment;

FIG. 11 is a diagram for explaining an outline of the determination method for determination performed by the determining unit that is included in the medical support apparatus according to one embodiment;

FIG. 12 is a diagram for explaining an outline of the determination method for determination performed by the determining unit that is included in the medical support apparatus according to one embodiment;

FIG. 13 is a diagram for explaining an outline of the determination method for determination performed by the determining unit that is included in the medical support apparatus according to one embodiment;

FIG. 14 is a diagram illustrating an example of an imaging-report screen that is displayed on a display unit 62 by a display controller that is included in the medical support apparatus according to one embodiment;

FIG. 15 is a flowchart illustrating an outline of an editing process in FIG. 5;

FIG. 16 is a diagram illustrating an example of the imaging-report screen that is displayed by the display unit included in the medical support apparatus according to one embodiment;

FIG. 17 is a diagram schematically illustrating a main part of a portion of the imaging-report screen that is displayed by the display unit included in the medical support apparatus according to one embodiment;

FIG. 18 is a diagram schematically illustrating a main part of a portion of the imaging-report screen that is displayed by the display unit included in the medical support apparatus according to one embodiment;

FIG. 19 is a diagram schematically illustrating a content of edit performed by an editing unit that is included in the medical support apparatus according to one embodiment;

FIG. 20 is a diagram illustrating another example of the imaging-report screen that is displayed by the display unit included in the medical support apparatus according to one embodiment;

FIG. 21 is a diagram illustrating an example of a report that is displayed by the display unit included in the medical support apparatus according to one embodiment;

FIG. 22 is a diagram illustrating an example of an image that is displayed by the display unit included in the medical support apparatus according to one embodiment;

FIG. 23 is a diagram illustrating an example of an image that is displayed by the display unit included in the medical support apparatus according to one embodiment;

FIG. 24 is a diagram illustrating an example of an image that is displayed by the display unit included in the medical support apparatus according to one embodiment;

FIG. 25 is a diagram illustrating an example of an image that is displayed by the display unit included in the medical support apparatus according to one embodiment; and

FIG. 26 is a flowchart illustrating an outline of a transmission process in FIG. 5.

### DETAILED DESCRIPTION

Modes for carrying out the present disclosure will be described in detail below with reference to the drawings. The present disclosure is not limited by the embodiments below. Further, each of the drawings referred to in the description below just schematically illustrates a shape, a size, and a positional relationship to understand details of the present disclosure. In other words, the present disclosure is not limited to the shape, the size, and the positional relationship illustrated in each of the drawings. Furthermore, in description of the drawings, the same components are denoted by the same reference symbols.

### Overall Configuration of Medical Support System

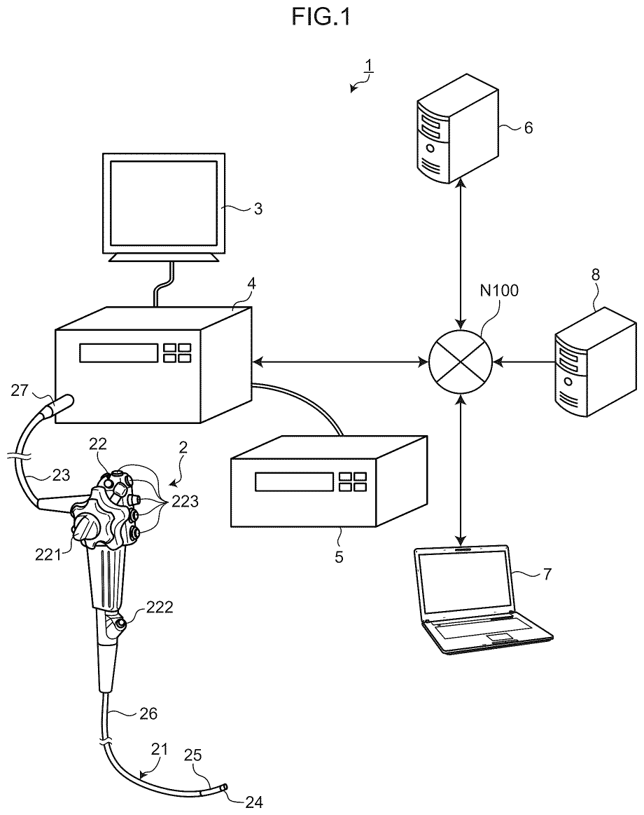

FIG. 1 is a diagram illustrating an overall configuration of a medical support system according to one embodiment. A medical support system 1 illustrated in FIG. 1 inserts an insertion portion 21 of an endoscope 2 from a mouth of a subject, such as a human being or an animal, to an esophagus or from an anus of the subject to a large intestine to consecutively capture images inside the subject and displays a group of captured image data on a display apparatus 3 in chronological order.

Further, in the medical support system 1, based on an image data group that is input from a control apparatus 4 and an inference model (trained model) that is trained in advance by using training data in which an organ, a region, a lesion, or the like of a subject is captured, an image diagnosis apparatus 5 (Computer Aided Detection (CAD)) detects (recognizes or estimates) an organ or a region in a plurality of observed regions that appear in the image data, a feature area including a polyp, a cancer, or the like, and treatment on a living body by a treatment tool, such as forceps or a snare, and outputs detection result information to the control apparatus 4. An operator, such as a doctor, performs observation and treatment on the subject while checking a display image displayed on the display apparatus 3 and a detection result (estimation result) of the image diagnosis apparatus 5.

Further, in the medical support system 1, a medical support apparatus 6 acquires an image file in which an image (still image data) that is input from the control apparatus 4 via a network N100 is stored, and the detection result information that includes the detection result (estimation result) of the image diagnosis apparatus 5, and records the acquired image file and the acquired detection result information in an associated manner. Furthermore, the medical support system 1 outputs various kinds of data in response to a request from a laptop computer 7 of the operator or the like via the network N100.

Moreover, in the medical support system 1, the medical support apparatus 6 or the laptop computer 7 creates report information, such as an examination report, on the subject by the operator or the like, and the report information is output and recorded to and by the medical support apparatus 6. Thereafter, the operator performs diagnosis, a conference, or the like based on the report information, a diagnosis result on a living cell that is acquired by biopsy at the time of observation of the subject, or the like.

Furthermore, in the medical support system 1, the medical support apparatus 6 transmits the image file or the report to an image management server 8 (PACS server), and the image management server 8 manages the image file or the report that is transmitted from each of the medical support apparatuses 6.

As illustrated in FIG. 1, the medical support system 1 includes the endoscope 2, the display apparatus 3, the control apparatus 4, the image diagnosis apparatus 5, and the medical support apparatus 6.

The endoscope 2 consecutively generates image data (RAW data) by capturing images inside the subject, and sequentially outputs pieces of the image data to the control apparatus 4. The endoscope 2 includes, as illustrated in FIG. 1, the insertion portion 21, an operating unit 22, and a universal cord 23.

The insertion portion 21 includes at least a part that has flexibility, and is inserted into the subject. The insertion portion 21 includes, as illustrated in FIG. 1, a distal end portion 24 that is arranged on a distal end of the insertion portion 21, a bending portion 25 that is connected to a proximal end side (at the side of the operating unit 22) of the distal end portion 24 and that is bendable, and a flexible tube portion 26 that is connected to a proximal end side of the bending portion 25 and that has flexibility.

The operating unit 22 is connected to a proximal end portion of the insertion portion 21. The operating unit 22 receives various kinds of operation on the endoscope 2. The operating unit 22 includes, as illustrated in FIG. 1, a bending knob 221, an insertion port 222, and a plurality of operating members 223.

The bending knob 221 is configured so as to rotatable in accordance with user operation performed by a user, such as an operator. Further, the bending knob 221, by rotating, causes a bending mechanism (not illustrated) that is arranged in the insertion portion 21 and that is made of metal or resin to operate. With this configuration, the bending portion 25 bends.

The insertion port 222 is an insertion port that communicates with a treatment tool channel (not illustrated) that is a pipeline extended from the distal end of the insertion portion 21 and that is used for inserting a treatment tool or the like to the treatment tool channel from outside of the endoscope 2.

The plurality of operating members 223 include a button or the like for receiving various kinds of operation performed by the user, such as the operator, and outputs operation signals corresponding to the various kinds of operation to the control apparatus 4 via the universal cord 23. Examples of the various kinds of operation include release operation of instructing the endoscope 2 to capture a still image and switching operation of changing an observation mode of the endoscope 2 to a normal light observation mode or a special observation mode.

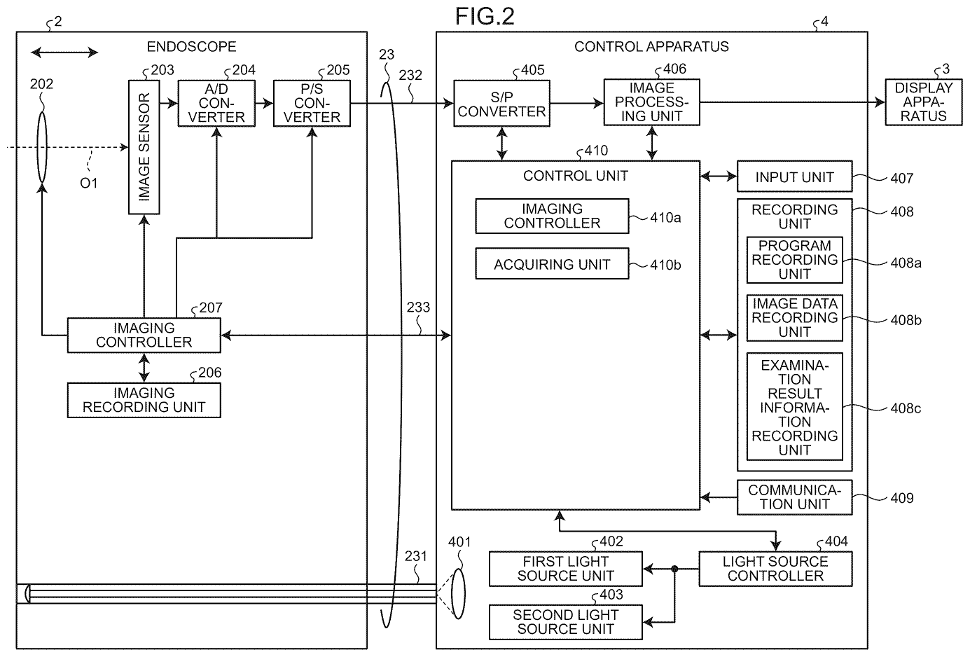

The universal cord 23 is a cord which is extended from the operating unit 22 in a different direction from an extending direction of the insertion portion 21, and in which a light guide 231 (see FIG. 2) configured with an optical fiber or the like, a first signal line 232 (see FIG. 2) for transmitting the above-described image data, a second signal line 233 (see FIG. 2) for transmitting the above-described operation signal, and the like are arranged. Further, as illustrated in FIG. 1, a first connector 27 is arranged on a proximal end of the universal cord 23. The first connector 27 is removably connected to the control apparatus 4.

The display apparatus 3 is configured with a display monitor made of liquid crystal, organic Electro Luminescence (EL), or the like, and displays an image based on image data subjected to image processing by the control apparatus 4 and various kinds of information on the endoscope 2, under the control of the control apparatus 4.

The control apparatus 4 is implemented by using a processor that is a processing apparatus including hardware, such as a Graphics Processing Unit (GPU), a Field Programmable Gate Array (FPGA), or a Central Processing Unit (CPU), and a memory that is a temporary storage area used by the processor. The control apparatus 4 comprehensively controls operation of each of the units of the endoscope 2 in accordance with a program that is recorded in the memory.

The image diagnosis apparatus 5 (CAD) detects (estimates), by using the image data group that is input from the control apparatus 4 and a trained model that is trained in advance by training data, an organ or a region that appears in image data, an insertion speed of the insertion portion 21, a feature area (an abnormal area or a candidate lesion area) including a polyp, a cancer, or the like, and treatment on a living body by a treatment tool, such as forceps or a snare, and outputs the detection result information in which a detection result and a detection time are associated to the control apparatus 4. The image diagnosis apparatus 5 is implemented by using a processor that is a processing apparatus including hardware, such as a GPU, an FPGA, or a CPU, and a memory that is a temporary storage area used by the processor.

The medical support apparatus 6 sequentially records various kinds of data input from the control apparatus 4 and recognition results of the image diagnosis apparatus 5, and transmits data in response to a request from the laptop computer 7. The medical support apparatus 6 is implemented by using a processor that is a processing apparatus including hardware, such as a GPU, an FPGA, or a CPU, and a memory that is a temporary storage area used by the processor. Further, the medical support apparatus 6 is configured with a Hard Disk Drive (HDD), a Solid State Drive (SSD), or the like.

### Functional Configuration of Main Part of Medical Support System

A functional configuration of a main part of the medical support system 1 as described above will be described below.

FIG. 2 is a block diagram illustrating functional configurations of main parts of the endoscope 2 and the control apparatus 4. In the following, the endoscope 2 and the control apparatus 4 will be described in this order, and thereafter, the image diagnosis apparatus 5 and the medical support apparatus 6 will be described.

### Functional Configuration of Endoscope

Firstly, a configuration of the endoscope 2 will be described.

As illustrated in FIG. 2, the endoscope 2 includes an illumination optical system 201, an imaging optical system 202, an image sensor 203, an analog-to-digital (A/D) converter 204, a parallel-to-serial (P/S) converter 205, an imaging recording unit 206, and an imaging controller 207. Here, each of the illumination optical system 201, the imaging optical system 202, the image sensor 203, the A/D converter 204, P/S converter 205, the imaging recording unit 206, and the imaging controller 207 is arranged in the distal end portion 24.

The illumination optical system 201 includes one or more lenses or the like, and emits illumination light supplied form the light guide 231 toward an imaging object.

The imaging optical system 202 includes a plurality of lenses, and an actuator that includes a stepping motor or a voice coil motor for causing a predetermined lens among the plurality of lenses to move in an optical axis direction. The imaging optical system 202 condenses reflected light from the imaging object, returning light from the imaging object, fluorescence generated by the imaging object, or the like, and forms an object image on a light receiving surface of the image sensor 203. Further, the imaging optical system 202 is able to change a focal distance (imaging magnification or an enlargement factor) and a focal position by causing a predetermined lens to move along an optical axis direction O1, under the control of the imaging controller 207. According to one embodiment, the imaging optical system 202 is able to change the imaging magnification to one-fold, 80-fold, or 520-fold. The imaging optical system 202 need not always change the imaging magnification in a stepwise manner, but may be configured to linearly change the imaging magnification.

The image sensor 203 is configured with an image sensor of a Charge Coupled Device (CCD) or a Complementary Metal Oxide Semiconductor (CMOS) in which one of color filters constituting Bayer arrangement (RGGB) is arranged on each of pixels that are arranged in a two-matrix manner. The image sensor 203 receives the object image that is formed by the imaging optical system 202, performs photoelectric conversion, and generates a captured image (analog signal), under the control of the imaging controller 207. Meanwhile, in the present embodiment, the image sensor 203 may be configured such that an image sensor and a TOF sensor that acquires imaging-object distance information (hereinafter, described as depth map information) by a Time Of Flight (TOF) method are integrated. The depth map information is information in which an imaging-object distance from a position of the image sensor 203 (position of the distal end portion 24) to a corresponding position on an observation object corresponding to a pixel position in the captured image is detected for each pixel position. Meanwhile, a component that generates the depth map information is not limited to the TOF sensor as described above, but it may be possible to adopt an image sensor or the like that includes a phase difference sensor. In the following, the depth map information and the captured image will be collectively described as image data. The image sensor 203 outputs the image data to the A/D converter 204.

The A/D converter 204 is configured with an A/D conversion circuit or the like. The A/D converter 204 performs an A/D conversion process on analog image data input from the image sensor 203 and outputs the converted image data to the P/S converter 205, under the control of the imaging controller 207.

The P/S converter 205 is configured with a P/S conversion circuit or the like, performs parallel to serial conversion on digital image data that is input from the A/D converter 204, and outputs the converted image data to the control apparatus 4 via the first signal line 232, under the control of the imaging controller 207.

Meanwhile, it may be possible to arrange, instead of the P/S converter 205, an electrical-to-optical (E/O) converter that converts the image data to an optical signal, and output the image data by the optical signal to the control apparatus 4. Further, for example, it may be possible to transmit the image data to the control apparatus 4 by radio communication by Wireless Fidelity (Wi-Fi) (registered trademark) or the like.

The imaging recording unit 206 may be configured with a non-volatile memory or a volatile memory, and records therein various kinds of information on the endoscope 2 (for example, pixel information on the image sensor 203). Further, the imaging recording unit 206 records therein various kinds of setting data and control parameters that are transmitted from the control apparatus 4 via the second signal line 233.

The imaging controller 207 is implemented by using a Timing Generator (TG), a processor that is a processing apparatus including hardware, such as a CPU, and a memory that is a temporary storage area used by the processor. The imaging controller 207 controls operation of each of the image sensor 203, the A/D converter 204, and the P/S converter 205 based on setting data that is received from the control apparatus 4 via the second signal line 233.

### Configuration of Control Apparatus

A configuration of the control apparatus 4 will be described below.

As illustrated in FIG. 2, the control apparatus 4 includes a condenser lens 401, a first light source unit 402, a second light source unit 403, a light source controller 404, an S/P converter 405, an image processing unit 406, an input unit 407, a recording unit 408, a communication unit 409, and a control unit 410.

The condenser lens 401 condenses light that is emitted by each of the first light source unit 402 and the second light source unit 403, and emits the condensed light to the light guide 231.

The first light source unit 402 emits white light (normal light) that is visible light and supplies the white light, as the illumination light, to the light guide 231, under the control of the light source controller 404. The first light source unit 402 is configured with a collimator lens, a white Light Emitting Diode (LED) lamp, a driving driver, and the like. Meanwhile, as the first light source unit 402, it may be possible to use a red LED lamp, a green LED lamp, and a blue LED lamp and cause the LED lamps to simultaneously emit light to supply the white light that is visible light. Furthermore, the first light source unit 402 may be configured with a halogen lamp, a xenon lamp, or the like.

The second light source unit 403 emits special light with a predetermined wavelength band and supplies the special light, as the illumination light, to the light guide 231, under the control of the light source controller 404. Here, the special light is light that is used for Narrow band Imaging (NBI) using narrow band light including 390 nm (nanometers) to 445 nm and 530 nm to 550 nm. It is of course possible to use, as the special light, amber light (600 nm and 630 nm) that is used for Red dichromatic Imaging (RDI), instead of the narrow band light.

The light source controller 404 is implemented by using a processor including hardware, such as an FPGA or a CPU, and a memory that is temporary storage area used by the processor. The light source controller 404 controls a light emission timing, a light emission time, and the like of each of the first light source unit 402 and the second light source unit 403 based on control data that is input from the control unit 410.

The S/P converter 405 performs serial to parallel conversion on the image data that is received from the endoscope 2 via the first signal line 232, and outputs the converted image data to the image processing unit 406, under the control of the control unit 410. Meanwhile, when the endoscope 2 outputs the image data by an optical signal, it may be possible to arrange an optical-to-electrical (O/E) converter that converts the optical signal to an electrical signal, instead of the S/P converter 405. Furthermore, when the endoscope 2 transmits the image data by radio communication, it may be possible to arrange a communication module that is able to receive a radio signal, instead of the S/P converter 405.

The image processing unit 406 is implemented by using a processor including hardware, such as a GPU or an FPGA, and a memory that is a temporary storage area used by the processor. The image processing unit 406 performs predetermined image processing on image data that is parallel data input from the S/P converter 405 and outputs the converted image data to the display apparatus 3, under the control of the control unit 410. Here, examples of the predetermined image processing include a demosaic process, a white balance process, a gain adjustment process, a γ correction process, and a format conversion process.

The input unit 407 is configured with a mouse, a foot switch, a keyboard, a button, a switch, a touch panel, or the like, receives user operation performed by a user, such as the operator, and outputs an operation signal corresponding to the user operation to the control unit 410.

The recording unit 408 is configured with a recording medium, such as a volatile memory, a non-volatile memory, a Solid State Drive (SSD), or a Hard Disk Drive (HDD). Further, the recording unit 408 records therein data including various kinds of parameters that are needed for operation of the control apparatus 4 and the endoscope 2. Further, the recording unit 408 includes a program recording unit 408a for recording various kinds of programs for operating the endoscope 2 and the control apparatus 4, an image data recording unit 408b for recording an image file that stores therein an image corresponding to image data, and an examination result information recording unit 408c for recording the detection result information.

The image data recording unit 408b records therein, under the control of the control unit 410, images (endoscopic images) corresponding to an image data group that is generated by the endoscope 2 by consecutively imaging a plurality of observed regions of the subject and corresponding to still image data, in association with patient information or the like. Here, the still image data is image data that is captured by the image sensor 203 when imaging is instructed by a release signal that is input from the operating unit 22, and is image data with higher resolution than live view images that are sequentially generated by the image sensor 203.

The examination result information recording unit 408c records therein the detection result information that is input from the image diagnosis apparatus 5, a detection time of the detection result, and patient information in an associated manner. Here, the detection result information includes a type of a feature area, size information (lesion size information) indicating a size of the feature area, region information on an organ or a region in the body of the subject at the time of capturing the image, and treatment information on treatment using a treatment tool. Here, the feature area is a lesion area including a cancer, a lesion, bleeding, or the like that is detected by the image diagnosis apparatus 5 (to be described later).

The communication unit 409, under the control of the control unit 410, transmits various kinds of information to the medical support apparatus 6 via the image diagnosis apparatus 5 and the network N100, receives various kinds of information from the image diagnosis apparatus 5 and the medical support apparatus 6, and outputs various kinds of information to the control unit 410. Specifically, the communication unit 409, under the control of the control unit 410, transmits temporally consecutive image data groups subjected to image processing by the image processing unit 406 to the image diagnosis apparatus 5, receives a detection result including an organ, a region, a feature area, and the like that is detected by the image diagnosis apparatus 5, and outputs the detection result to the control unit 410. Further, the communication unit 409, under the control of the control unit 410, transmits detection result information including the image data group (moving image data), the image data (still image data), and the detection result of the image diagnosis apparatus 5 to the medical support apparatus 6, and receives patient information on the subject, observation information indicating an observation order of observed portions at the time of examination on the subject, and the like from the medical support apparatus 6. The communication unit 409 is configured with a communication module or the like.

The control unit 410 corresponds to a second processor according to the present disclosure. The control unit 410 is implemented by using a second processor that is a processing apparatus including hardware, such as an FPGA or a CPU, and a memory that is a temporary storage area used by the second processor. Further, the control unit 410 comprehensively controls each of the units included in the endoscope 2 and the control apparatus 4. The control unit 410 includes an imaging controller 410a and an acquiring unit 410b.

The imaging controller 410a controls imaging of the endoscope 2. The imaging controller 410a causes the endoscope 2 to consecutively perform imaging and generate temporally consecutive image data groups. Further, when the release signal is input from the operating unit 22, the imaging controller 410a causes the image sensor 203 to capture still image data.

The acquiring unit 410b acquires the image data from the endoscope 2 via the S/P converter 405 or the image processing unit 406, and records the image data in the image data recording unit 408b. Further, the acquiring unit 410b acquires the detection result information from the image diagnosis apparatus 5 via the communication unit 409, and records the detection result information in the examination result information recording unit 408c.

### Functional Configuration of Image Diagnosis Apparatus

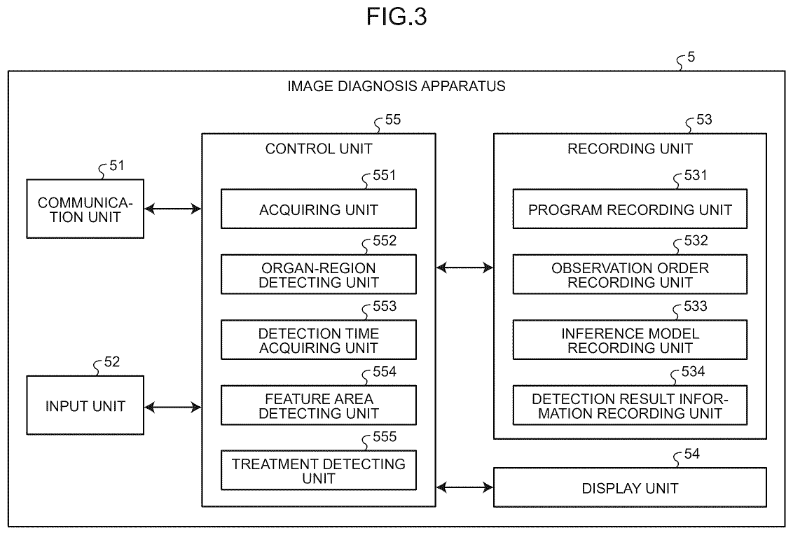

A functional configuration of the image diagnosis apparatus 5 will be described below. FIG. 3 is a block diagram illustrating the functional configuration of the image diagnosis apparatus 5. The image diagnosis apparatus 5 illustrated in FIG. 3 includes a communication unit 51, an input unit 52, a recording unit 53, a display unit 54, and a control unit 55.

The communication unit 51, under the control of the control unit 55, receives the image data group that is generated by the endoscope 2 and organ-region order information that indicates an observation order of organs and regions in the subject who is a patient from the control apparatus 4 via the network N100, and outputs the image data group and the organ-region order information to the control unit 55. Further, the communication unit 51, under the control of the control unit 55, outputs the detection result information including the detection result in which an organ or the like of the subject is detected and which is input from the control unit 55 and a detection time at which the detection result is detected to the control apparatus 4 via the network N100. The communication unit 51 is configured with a communication module or the like.

The input unit 52 is configured with a button, a switch, a touch panel, or the like, receives various kinds of input, and performs various kinds of output to the control unit 55.

The recording unit 53 is configured with a volatile memory, a non-volatile memory, an SSD, an HDD, or a recording medium, such as a memory card. The recording unit 53 records therein data that includes various kinds of parameters or the like needed for operation of the image diagnosis apparatus 5. Further, the recording unit 53 includes a program recording unit 531 for recording various kinds of programs for operating the image diagnosis apparatus 5, an observation order recording unit 532, an inference model recording unit 533, and a detection result information recording unit 534.

The observation order recording unit 532 records therein observation order information which is input from the medical support apparatus 6 and in which an observation order of a plurality of observation portions, such as organs and regions, that are set in advance is set at the time of observation of the subject by the endoscope 2.

The inference model recording unit 533 records therein an inference model that is used to detect an organ, a region, and a feature area based on the image data. Here, the inference model receives image data of the endoscope 2 as input and outputs an organ, a region, and a feature area included in the image data as output. The inference model is a model that is generated by machine learning using, for example, Artificial Intelligence (AI). Specifically, the inference model is a model that is trained by using teacher data that is obtained by adding a feature area, such as an organ, a region, and a lesion, of the subject, a treatment tool, and treatment to image data in which the subject is captured, and outputs the organ, the region, the feature area included in the image data, a size of the feature area, the treatment tool, and a treatment content.

The detection result information recording unit 534 records therein the detection result information that is detected by the image diagnosis apparatus 5, the detection time of the detection result, and the patient information in an associated manner.

The display unit 54 displays various kinds of information on the image diagnosis apparatus 5 under the control of the control unit 55. The display unit 54 is configured with a liquid crystal display, an organic Electro Luminescence (EL) display, or the like.

The control unit 55 is implemented by using a processor including hardware, such as a and FPGA or a CPU, and a memory that is a temporary storage area used by the processor. The control unit 55 comprehensively controls each of the units included in the medical support apparatus 6. The control unit 55 includes an acquiring unit 551, an organ-region detecting unit 552, and a detection time acquiring unit 553.

The acquiring unit 551 chronologically acquires pieces of image data that are sequentially generated by the endoscope 2, from the control apparatus 4 via the communication unit 51.

The organ-region detecting unit 552 detects an organ or a region of the subject included in the image data based on the pieces of image data that are sequentially acquired by the acquiring unit 551 and the inference model that is recorded in the inference model recording unit 533, and records the detection result as examination result information in the detection result information recording unit 534.

The detection time acquiring unit 553 acquires a detection time at which the organ-region detecting unit 552, a feature area detecting unit 554 (to be described later), and a treatment detecting unit 555 (to be described later) detects the organ or the region of the subject included in the image data, the feature area, and the treatment. For example, the detection time acquiring unit 553 acquires a time that is measured by a TG (not illustrated) as a detection time when the organ-region detecting unit 552 detects an organ or region of the subject included in the image data.

The feature area detecting unit 554 detects the feature area included in the image data and a size of the feature area based on the pieces of image data that are sequentially acquired by the acquiring unit 551 and the inference model that is recorded in the inference model recording unit 533, and records the detection result as the examination result information in the detection result information recording unit 534. Here, the feature area is a lesion, such as a cancer, bleeding, or a polyp.

The treatment detecting unit 555 detects, as the treatment information, a type of a treatment tool included in the image data and treatment on a living body by the treatment tool based on the pieces of image data that are sequentially acquired by the acquiring unit 551 and the inference model that is recorded in the inference model recording unit 533, and records the detection result as the examination result information in the detection result information recording unit 534. Here, the treatment on the living body includes acquisition of a living body by biopsy, excision of a living body, or thermal treatment on a living body, and, in addition to this, medical practice, such as saline injection into a living body, may be included.

### Functional Configuration of Medical Support Apparatus

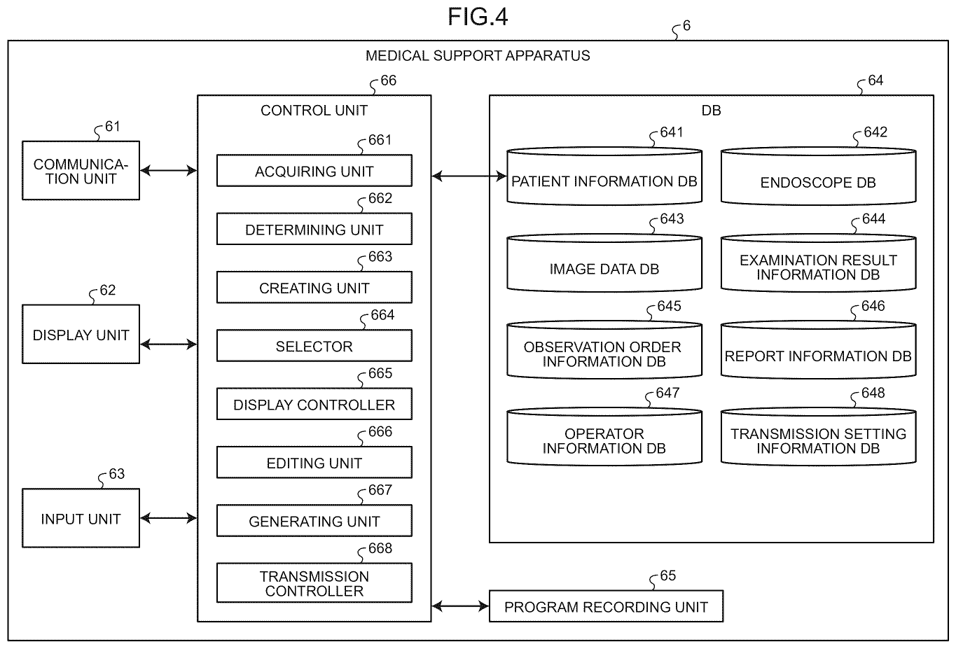

A functional configuration of the medical support apparatus 6 will be described below. FIG. 4 is a block diagram illustrating the functional configuration of the medical support apparatus 6. The medical support apparatus 6 illustrated in FIG. 4 includes a communication unit 61, a display unit 62, an input unit 63, a database 64 (hereinafter, simply described as a “DB 64”), a program recording unit 65, and a control unit 66.

The communication unit 61 transmits, under the control of the control unit 66, various kinds of information to the control apparatus 4 and the image diagnosis apparatus 5 via the network N100, and receives various kinds of information from the control apparatus 4 and the image diagnosis apparatus 5. The communication unit 61 is configured with a communication module.

The display unit 62 displays various kinds of information on the medical support apparatus 6 under the control of the control unit 66. The display unit 62 is configured with a liquid crystal display, an organic Electro Luminescence (EL) display, or the like.

The input unit 63 receives various kinds of input from operation performed from outside, and outputs the received input to the control unit 66. The input unit 63 is configured with a button, a keyboard, a switch, a touch panel, or the like.

The DB 64 records therein various kinds of information. The DB 64 is configured with an HDD, an SSD, or the like. The DB 64 includes a patient information DB 641, an endoscope DB 642, an image data DB 643, an examination result information DB 644, an observation order information DB 645, a report information DB 646, an operator information DB 647, and a transmission setting information DB 648.

The patient information DB 641 records therein, for each patient, a patient ID for identifying a patient, a date of birth of the patient, a primary doctor of the patient, an address, and allergy information on the patient.

The endoscope DB 642 records therein, for each of the endoscopes 2, a type of the endoscope 2, an endoscope ID, a repair history of the endoscope 2, a maintenance status (maintenance condition) of the endoscope 2, cleaning history information on a cleaning history of the endoscope 2, a usage history of the endoscope 2, and scheduled usage information.

The image data DB 643 records therein, for each patient ID or each operator ID, image data groups captured by the endoscope 2, in an associated manner.

The examination result information DB 644 records therein, for each patient ID, a past examination result. Further, the examination result information DB 644 records therein the detection result information on the plurality of organs, regions, a feature area, and the treatment information on the subject that are detected by the image diagnosis apparatus 5 based on the image data groups captured by the endoscope 2, in association with the examination result of each patient ID.

The observation order information DB 645 records therein an observation order of organs and regions by the endoscope 2, which is set for each facility ID or each operator ID.

The report information DB 646 records therein, for each patient ID or each operator ID, report information that is generated by an operator (observer) by the endoscope 2 and report information that is automatically generated by the medical support apparatus 6, in an associated manner.

The operator information DB 647 records therein, for each operator ID, a name, a job title, affiliation, and contact information, in an associated manner.

The transmission setting information DB 648 records therein various kinds of setting information that are set by a user in advance when the medical support apparatus 6 transmits an image to the image management server 8. Specifically, the transmission setting information DB 648 records therein, for example, setting information for transmitting only a predetermined image, such as a representative image, from among a plurality of images when the medical support apparatus 6 transmits an image to the image management server 8.

The program recording unit 65 is configured with a volatile memory, a non-volatile memory, an SSD, or the like, and records therein various kinds of programs for operating the medical support apparatus 6.

The control unit 66 corresponds to a first processor according to the present disclosure. The control unit 66 is implemented by using a first processor including hardware, such as an FPGA or a CPU, and a memory that is a temporary storage area used by the first processor. Further, the control unit 66 comprehensively controls each of the units included in the medical support apparatus 6. The control unit 66 includes an acquiring unit 661, a determining unit 662, a creating unit 663, a selector 664, a display controller 665, an editing unit 666, a generating unit 667, and a transmission controller 668.

The acquiring unit 661 acquires a plurality of image files from the control apparatus 4 and the examination result information from the image diagnosis apparatus 5 via the network N100. The acquiring unit 661 records the plurality of image files in the image data DB 643 and records the examination result information in the examination result information DB 644.

The determining unit 662 determines the plurality of images that include identical features areas as an identical group based on imaging magnification information included in the plurality of image files acquired by the acquiring unit 661 and the detection result information. Specifically, the determining unit 662 determines images including the identical feature areas (identical lesions) and classifies images with the identical feature areas (identical lesions) as an identical group. More specifically, the determining unit 662 adopts a time of an image for which imaging magnification is one-fold magnification that indicates a non-enlarged image among the plurality of images as a start point, adopts a time of another image for which imaging magnification is one-fold magnification that indicates a non-enlarged image among the plurality of images as an end point, and determines a plurality of enlarged images (imaging magnification of two or more) in the adopted period as images with identical feature areas. Furthermore, the determining unit 662 determines a group of images including identical lesions as identical feature areas, that is, identical lesion images based on an imaging time, region information, and a lesion size. Specifically, the determining unit 662 determines images that are temporally consecutive non-enlarged images with one-fold magnification, that are captured within a predetermined period (for example, within one minute), and that have identical imaging regions and identical lesion sizes as identical lesion images in which identical feature areas, that is, identical lesions are captured. Moreover, when a non-enlarged image with one-fold magnification is obtained after an image with largest imaging magnification is obtained, and if imaging times are within a predetermined period, if regions are identical, and if treatment is performed, the determining unit 662 determines a group of the obtained images as identical lesion images in which identical feature areas, that is, identical lesions are captured.

The creating unit 663 creates an image group for each feature area based on a determination result determined by the determining unit 662. Specifically, the creating unit 663 creates an image group for each lesion based on a determination result obtained by determination performed by the determining unit 662.

The selector 664 selects a representative image for each of the groups created by the creating unit 663, based on the imaging magnification information. Specifically, when the group includes a plurality of images with same imaging magnification, the selector 664 selects, for example, an image with earliest imaging time as the representative image. Further, when the same group includes a plurality of images with different imaging magnification, the selector 664 selects a representative image for each imaging magnification.

The display controller 665 displays, on the display unit 62, an imaging-report screen including the representative image for each group selected by the selector 664.

The editing unit 666 performs display and editing in accordance with the content of operation performed by the user on the input unit 63. Specifically, the editing unit 666 performs editing in accordance with the content of operation performed by the user on the input unit 63, such as association of a group number with a predetermined region in a schematic diagram (to be described later) with respect to a group number field.

The generating unit 667 generates a report in accordance with the content of the operation performed by the user on the input unit 63. Specifically, the generating unit 667 receives input of diagnosis information on a content of diagnosis performed by the user on the subject in accordance with the content of operation performed by the user, registers the received diagnosis information, and generates a report in which the diagnosis information is reflected.

The transmission controller 668 deletes images other than the representative image of each of the groups from the image data DB 643, and transmits only the representative image of each of the groups to the image management server 8. Further, the transmission controller 668 transmits all of the images including the representative image and a still image group of each of the groups to the image management server 8.

Process performed by medical support apparatus

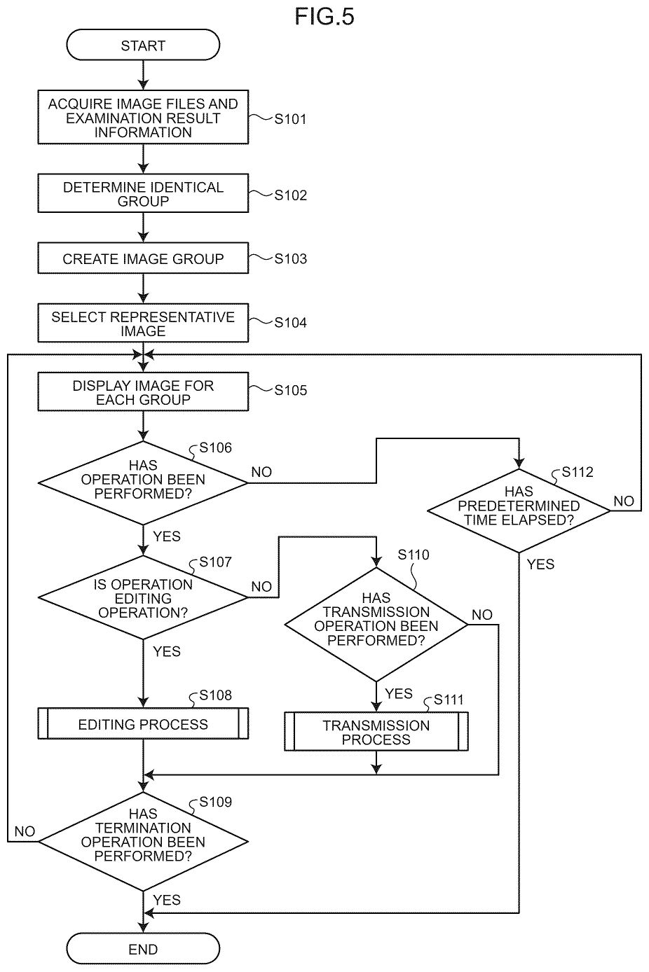

An outline of a process that is performed by the medical support apparatus 6 will be described below. FIG. 5 is a flowchart illustrating the outline of the process that is performed by the medical support apparatus 6.

As illustrated in FIG. 5, firstly, the acquiring unit 661 acquires a plurality of image files from the control apparatus 4 and the examination result information from the image diagnosis apparatus 5 (Step S101).

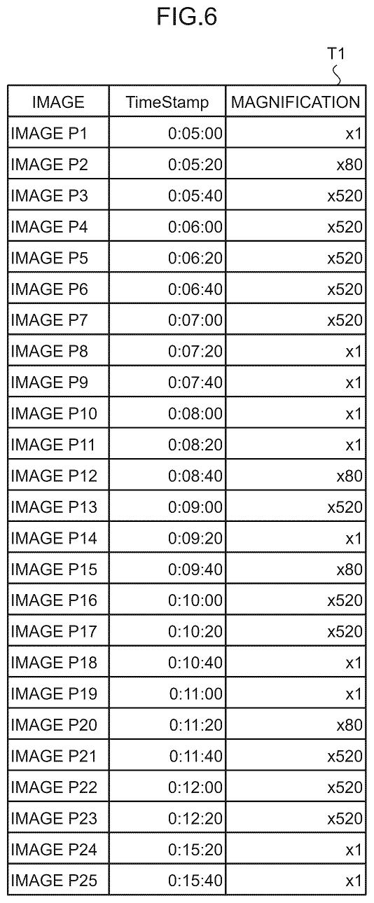

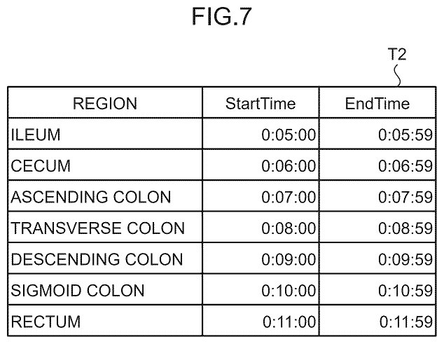

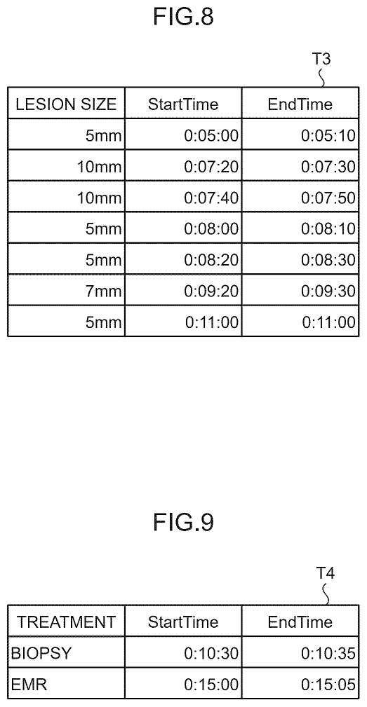

Details of the image files and the examination result information that are acquired by the acquiring unit 661 will be described below. FIG. 6 is a diagram illustrating an example of contents of each of the image files. FIG. 7 is a diagram illustrating an example of region information that is included in the examination result information. FIG. 8 is a diagram illustrating an example of lesion size information that is about a feature area and that is included in the examination result information. FIG. 9 is a diagram illustrating an example of treatment information that is included in the examination result information.

In an image file table T1 illustrated in FIG. 6, an image (still image data), TimeStamp that indicates an imaging time of the endoscope 2, and magnification that indicates the imaging magnification information on the endoscope 2 are recorded in an associated manner for each of the image files. Specifically, TimeStamp of “0:05:00” and magnification of “×1” are recorded in association with an image of “image P1”. Meanwhile, the image is still image data, such as JPEG data or RAW data, that is captured by the endoscope 2. Further, the magnification of “×1” indicates a non-enlarged image.

Furthermore, in a region information table T2 illustrated in FIG. 7, StartTime that indicates a detection start time and EndTime that indicates a detection end time are recorded in an associated manner for each organ or each region. Specifically, StartTime of “0:05:00” and EndTime of “0:05:59” are recorded in association with a region of “ileum”.

Moreover, in a lesion size table T3 illustrated in FIG. 8, StartTime that indicates a detection start time and EndTime that indicates a detection end time are recorded in an associated manner for each lesion size (hereinafter, simply described as a “lesion size”) that indicates a feature area size in a feature area. Specifically, StartTime of “0:05:00” and EndTime of “0:05:10” are recorded in association with a lesion size of “5 mm”.

Furthermore, in a processing information table T4 illustrated in FIG. 9, StartTime that indicates a detection start time and EndTime that indicates a detection end time are recorded in an associated manner for each treatment. Specifically, StartTime of “0:10:30” and EndTime of “0:10:35” are recorded in association with treatment of “biopsy”.

Referring back to FIG. 5, explanation from Step S102 will be continued.

At Step S102, the determining unit 662 determines images that include identical feature areas as an identical group, based on the imaging magnification information included in the plurality of image files and the detection result information that are acquired by the acquiring unit 661 (Step S102).

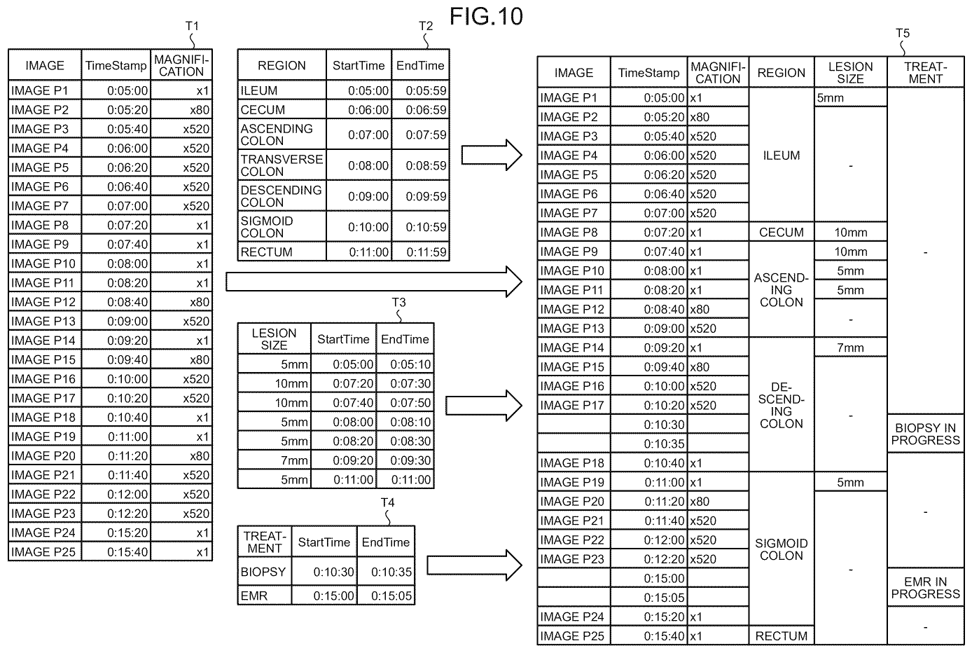

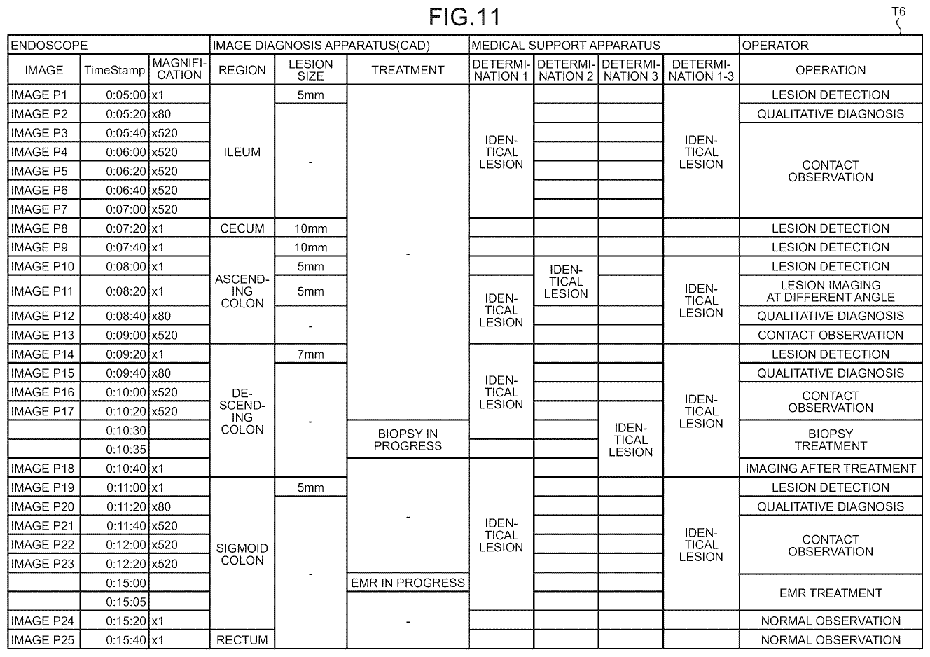

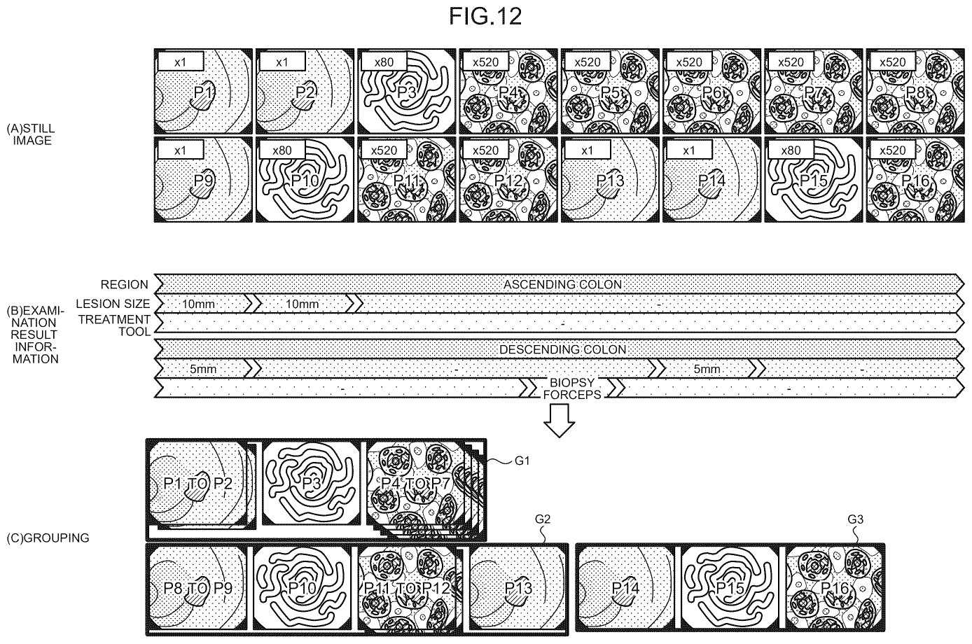

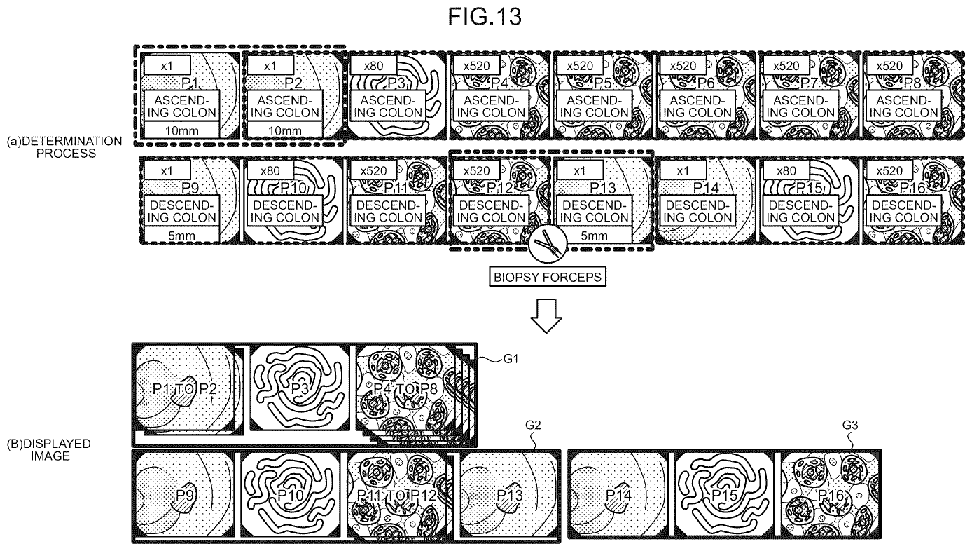

FIG. 10 is a diagram for explaining an outline of a determination method for determination that is performed by the determining unit 662. FIG. 11 is a diagram for explaining an outline of the determination method for determination that is performed by the determining unit 662. FIG. 12 is a diagram for explaining an outline of the determination method for determination that is performed by the determining unit 662. FIG. 13 is a diagram for explaining an outline of the determination method for determination that is performed by the determining unit 662. Meanwhile, numbers assigned to the images in FIG. 10 to FIG. 13 are mere examples, and the imaging magnification, the organ or the region of the subject, details of the treatment, or the like may be different among the drawings.

As illustrated in FIG. 10, firstly, the determining unit 662 generates an integrated information table T5 by performing integration on the imaging magnification information included in the plurality of image files, and the region information, the lesion size information, and the treatment information included in the detection result information, based on a detection time of each information acquired, where the plurality of image files and the detection result information are acquired by the acquiring unit 661. Specifically, as illustrated in FIG. 10, the determining unit 662 performs alignment and integration on the images in order from the earliest time of TimeStamp, and records (stores) the detection result information with the same time in a row of the image with the same time in an associated manner, so that the integrated information table T5 is generated.

Subsequently, as illustrated in FIG. 11 to FIG. 13, the determining unit 662 determines an identical group with an identical feature area (identical lesion region) by using one or more of a first determination method to a third determination method to be descried below. Specifically, the determining unit 662 determines images each including an identical feature area (identical lesion), and classifies each of the images for each identical group including an identical feature area (identical lesion).

### First Determination Method

The first determination method will be described below. In the case as illustrated in FIG. 11 to FIG. 13, the determining unit 662 adopts a time of an image for which the imaging magnification is one-fold magnification (×1) that indicates a non-enlarged image among the image P1 to an image P25 as a start point, adopts a time of another image for which the imaging magnification is one-fold magnification (×1) that indicates a non-enlarged image among the image P1 to an image P25 as an end point, and determines a plurality of enlarged images (with the imaging magnification of 2 or more) in the adopted period as images that include identical feature areas. Specifically, the determining unit 662 determines an image group that includes an identical lesion as an identical lesion image based on the imaging magnification information.

Specifically, in the case as illustrated in FIG. 11, the determining unit 662 adopts a time (0:05:00) of the image P1 for which the imaging magnification is one-fold magnification (×1) that indicates a non-enlarged image as a start point, adopts a time (0:0:7:40) of the image P9 for which the imaging magnification is one-fold magnification (×1) that indicates a non-enlarged image as an end point, and determines the image P1 to the image P7 that are a plurality of enlarged images in the adopted period as images including the identical feature areas. In other words, the determining unit 662 determines the image P1 to the image P7 as identical lesion images. Similarly, in the case as illustrated in FIG. 11, the determining unit 662 determines that each of a first group of an image P11 to an image P13, a second group of an image P14 to an image P17, and a third group of an image P19 to an image P23 has images that include identical feature areas, that is, each group has identical lesion images, but the first to third groups have images with different lesions in the subject between these groups.

Furthermore, in the case as illustrated in FIG. 12 and FIG. 13, the determining unit 662 determines that each of a first group of the image P1 to an image P8, a second group of an image P9 to an image P12, and a third group of an image P14 to an image P16 has images that include identical feature areas, that is, each group has identical lesion images, but the first to third groups have images with different lesions in the subject between these groups.

### Second Determination Method

The second determination method will be described below. As illustrated in FIG. 11 to FIG. 13, the determining unit 662 determines an image group that includes an identical lesion as an identical feature area, that is, an identical lesion image, based on the imaging time, the region information, and the lesion size.

Specifically, in the case as illustrated in FIG. 11, the determining unit 662 determines an image P10 and an image P11 that are temporally consecutive non-enlarged images with one-fold magnification (×1), that are captured within a predetermined period (for example, within one minute), and that have identical imaging regions (ascending colon) and identical lesion sizes (5 mm) as images that include identical feature areas. In other words, the determining unit 662 determines the image P10 and the image P11 as identical lesion images in which identical lesions are captured.

Furthermore, in the case as illustrated in FIG. 12 and FIG. 13, the determining unit 662 determines the image P1 and the image P2 that have identical imaging regions (ascending colon) and identical lesion sizes (5 mm) as images that include identical feature areas, that is, identical lesion images in which identical lesions are captured.

### Third Determination Method

The third determination method will be described below. As illustrated in FIG. 11 to FIG. 13, when a non-enlarged image with one-fold magnification is obtained after an image with maximum imaging magnification is obtained, and if imaging times are within a predetermined period, if regions are identical, and if treatment is performed, the determining unit 662 determines a group of the images as images that include identical feature areas.

Specifically, as illustrated in FIG. 11 to FIG. 13, the determining unit 662 determines the image P17 and an image P18 as images that include identical feature areas. Specifically, the determining unit 662 determines the image P17 and the image P18 as identical lesion images in which identical lesions (feature areas) are captured.

Furthermore, in the case as illustrated in FIG. 12 and FIG. 13, the determining unit 662 determines the image P12 and the image P13 in which biological treatment is performed as images that include identical feature areas, that is, identical lesion images in which identical lesions (feature areas) are captured.

In this manner, the determining unit 662 performs determination on a plurality of images included in a plurality of image files using any one or more of the first determination method to the third determination method and classifies the plurality of images included in the plurality of image files according to identical feature area, that is, identical lesion image.

Referring back to FIG. 5, explanation from Step S103 will be described.

At Step S103, the creating unit 663 creates an image group for each feature area based on a determination result that is obtained by the determination performed by the determining unit 662. Specifically, in the case as illustrated in FIG. 12 and FIG. 13 as described above, the creating unit 663 creates a group G1 for the image P1 to the image P8, a group G2 for the image P9 to the image P13, and a group G3 for the image P14 to the image P16 with respect to the image P1 to the image P16 based on the determination result that is obtained by the determination performed by the determining unit 662.

Subsequently, the selector 664 selects a representative image for each of the groups created by the creating unit 663 based on the imaging magnification information (Step S104). Specifically, when each of the groups that are created by the creating unit 663 includes images with the same imaging magnification, the selector 664 selects an image at the earliest imaging time as the representative image. More specifically, as illustrated in FIG. 12 and FIG. 13, when the group G1 that is created by the creating unit 663 includes images with the same imaging magnification, the selector 664 selects the image P1 at the earliest imaging time as a representative image for the non-enlarged images and selects the image P4 at the earliest imaging time as a representative image for the images with the imaging magnification of 520-fold (×520). It is of course possible for the selector 664 to select an image at the latest imaging time or at an intermediate time among images with the same magnification as the representative image, and, when a plurality of images with the same magnification are present, it may be possible to select the representative image in accordance with a setting content that is preset by the operator on the medical support apparatus 6 or select a most-focused image as the representative image.

Thereafter, the display controller 665 displays an imaging-report screen including the representative image for each of the groups selected by the selector 664 on the display unit 62 (Step S105).

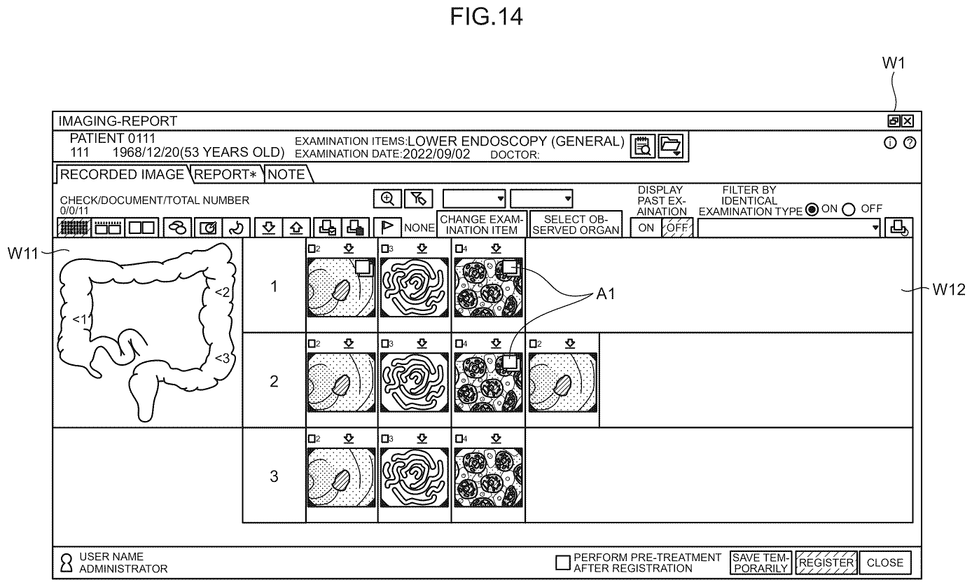

The imaging-report screen that is displayed on the display unit 62 will be described in detail below. FIG. 14 is a diagram illustrating an example of the imaging-report screen that is displayed on the display unit 62 by the display controller 665. As illustrated in FIG. 14, an imaging-report screen W1 includes a schema diagram W11 that schematically illustrates an organ or a region in the body of the subject, and a display area W12 in which the representative image for each of the groups determined as the identical feature areas is displayed.

In the case as illustrated in FIG. 14, the display controller 665 displays the representative image of each of the groups in the display area W12. Further, the display controller 665 displays a position in the organ in the body of the subject in which each of the groups is captured and a number in an associated manner. Specifically, the display controller 665 displays, as the group G1, a group that is captured at a position with the number of “1” in the schema diagram W11, displays, as the group G2, a group that is captured at a position with the number of “2” in the schema diagram W11, and displays, as the group G3, a group that is captured at a position with the number of “3” in the schema diagram W11. Furthermore, the display controller 665 displays an icon A1 indicating that a plurality of images that are captured with the same imaging magnification are present in addition to the representative image, in a superimposed manner on the representative image.

Referring back to FIG. 5, explanation from Step S106 will be described.

At Step S106, the determining unit 662 determines whether or not the user has performed operation on the input unit 63. When the determining unit 662 determines that the user has performed operation on the input unit 63 (Step S106: Yes), the medical support apparatus 6 goes to Step S107 to be described later. In contrast, when the determining unit 662 determines that the user has not performed operation on the input unit 63 (Step S106: No), the medical support apparatus 6 goes to Step S112 to be described later.

At Step S107, the determining unit 662 determines whether or not the operation performed by the user on the input unit 63 is editing operation. When the determining unit 662 determines that the operation performed by the user on the input unit 63 is the editing operation (Step S107: Yes), the medical support apparatus 6 goes to Step S108 to be described later. In contrast, when the determining unit 662 determines that the operation performed by the user on the input unit 63 is not the editing operation (Step S107: No), the medical support apparatus 6 goes to Step S110 to be described later.

At Step S108, the medical support apparatus 6 performs an editing process of associating an image group for each group with the schema diagram that schematically illustrates an organ based on details of the operation performed by the user on the input unit 63. Meanwhile, details of the editing process will be described later. After Step S108, the medical support apparatus 6 goes to Step S109 as described below.

At Step S109, the determining unit 662 determines whether or not the user has performed termination operation on the input unit 63. When the determining unit 662 determines that the user has performed the termination operation on the input unit 63 (Step S109: Yes), the medical support apparatus 6 terminates the process. In contrast, when the determining unit 662 determines that the user has not performed the termination operation on the input unit 63 (Step S109: No), the medical support apparatus 6 returns to Step S105 as described above.

At Step S110, the determining unit 662 determines whether or not the user has performed, on the input unit 63, transmission operation of transmitting an image to the image management server 8. When the determining unit 662 determines that the user has performed, on the input unit 63, the transmission operation of transmitting an image to the image management server 8 (Step S110: Yes), the medical support apparatus 6 goes to Step S111 to be described later. In contrast, when the determining unit 662 determines that the user has not performed, on the input unit 63, the transmission operation of transmitting an image to the image management server 8 (Step S110: No), the medical support apparatus 6 goes to Step S109.

At Step S111, the medical support apparatus 6 performs a transmission process of transmitting an image corresponding to the operation that is performed by the user on the input unit 63 to the image management server 8. Meanwhile, details of the transmission process will be described later. After Step S111, the medical support apparatus 6 goes to Step S109.

At Step S112, the determining unit 662 determines whether or not a predetermined time, for example, five minutes, has elapsed since display a group of the plurality of images. When the determining unit 662 determines that the predetermined time has elapsed since display of the group of the plurality of images (Step S112: Yes), the medical support apparatus 6 terminates the process. In contrast, when the determining unit 662 determines that the predetermined time has not elapsed since display of the group of the plurality of images (Step S112: No), the medical support apparatus 6 returns to Step S105 as described above.

### Outline of Editing Process

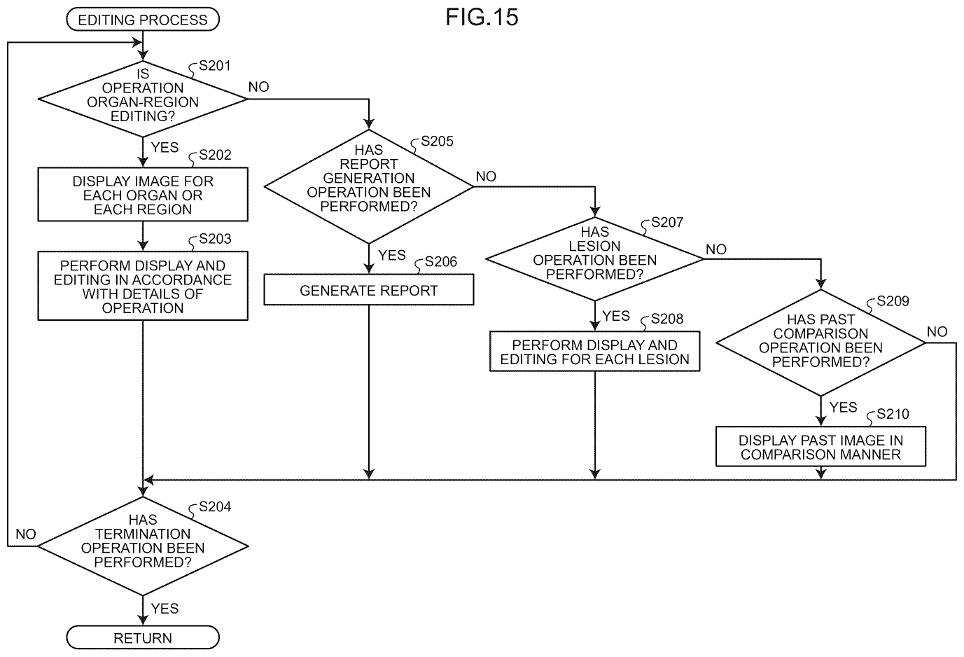

Details of the editing process described at Step S108 in FIG. 5 will be described in detail below. FIG. 15 is a flowchart illustrating an outline of the editing process.

As illustrated in FIG. 15, the determining unit 662 determines whether or not the editing operation is organ-region editing for associating an image with the organ or the region (Step S201).

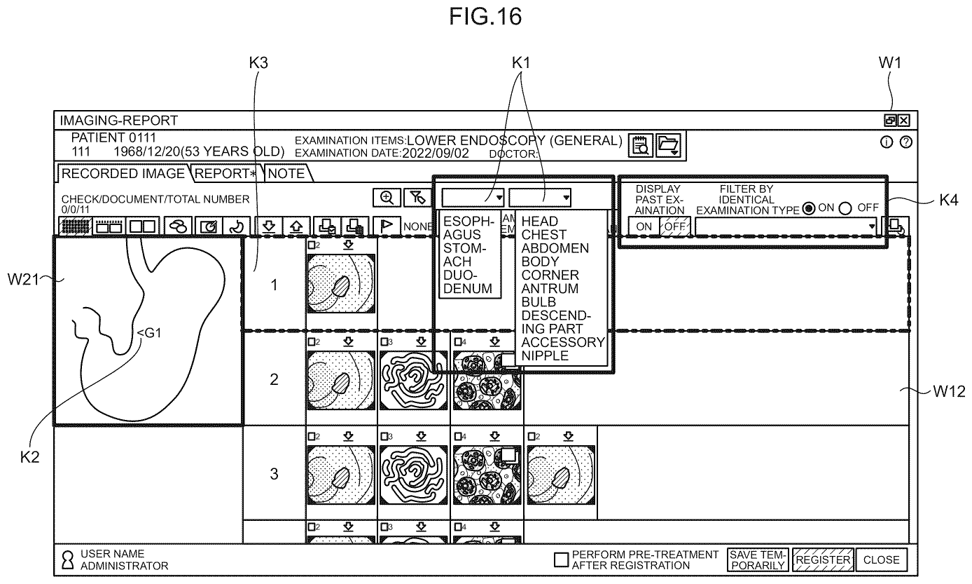

FIG. 16 is a diagram illustrating an example of an imaging-report screen that is displayed by the display unit 62. As illustrated in FIG. 16, the imaging-report screen W1 includes a cell K1 for receiving input of an instruction signal for displaying an image corresponding to a selected and desired organ or region, a button K2 for receiving input of an instruction signal for adding the group number to the report, a cell K3 for receiving input of an instruction signal for searching for and displaying the group number, and a cell region K4 for receiving input of an instruction signal for displaying a past group image of the same subject. The determining unit 662 determines whether or not the user has operated the input unit 63 and selected a desired organ or a desired region from the cell K1 in the imaging-report screen W1 displayed by the display unit 62 by, and when the desired organ or the desired region is selected in the cell K1, the determining unit 662 determines that the editing operation is the organ-region editing for associating an image with the organ or the region. When the determining unit 662 determines that the editing operation is the organ-region editing for associating an image with the organ or the region (Step S201: Yes), the medical support apparatus 6 goes to Step S203 to be described later. In contrast, when the determining unit 662 determines that the editing operation is not the organ-region editing for associating an image with the organ or the region (Step S201: No), the medical support apparatus 6 goes to Step S205 to be described later.

At Step S202, the display controller 665 displays an image for each organ or each region for each group associated with the organ or the region that is selected in the cell K1 in accordance with details of operation performed by the user on the input unit 63.

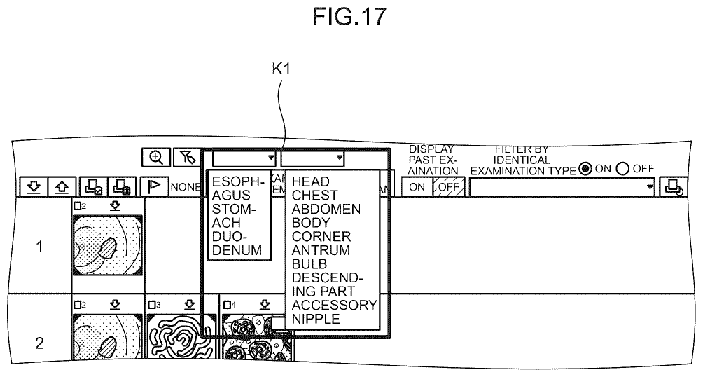

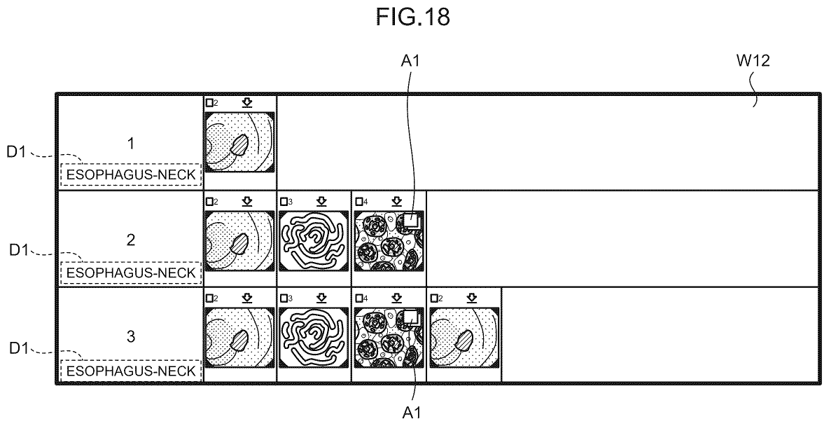

FIG. 17 is a diagram schematically illustrating a main part of a portion of the imaging-report screen that is displayed by the display unit 62. FIG. 18 is a diagram schematically illustrating a main part of a portion of the imaging-report screen that is displayed by the display unit 62.

As illustrated in FIG. 17, when the user operates the input unit 63 and selects a desired organ or a desired region from among a plurality of organs or a plurality of regions that are displayed in the cell K1, the display controller 665 displays descriptions D1 to D3 of an organ or a region for each group that is associated with the organ or the region selected in the cell K1 as illustrated in FIG. 18 (FIG. 17 to FIG. 18). In this case, the display controller 665 displays the description of each organ or each region in a superimposed manner on the group number. For example, as illustrated in FIG. 18, the display controller 665 displays “esophagus-neck” in a superimposed manner on the group number of “1”. With this configuration, the user is able to check an image of the desired organ or the desired region.

Referring back to FIG. 15, explanation from Step S203 will be described.

At Step S203, the editing unit 666 performs display and editing in accordance with details of operation performed by the user on the input unit 63.

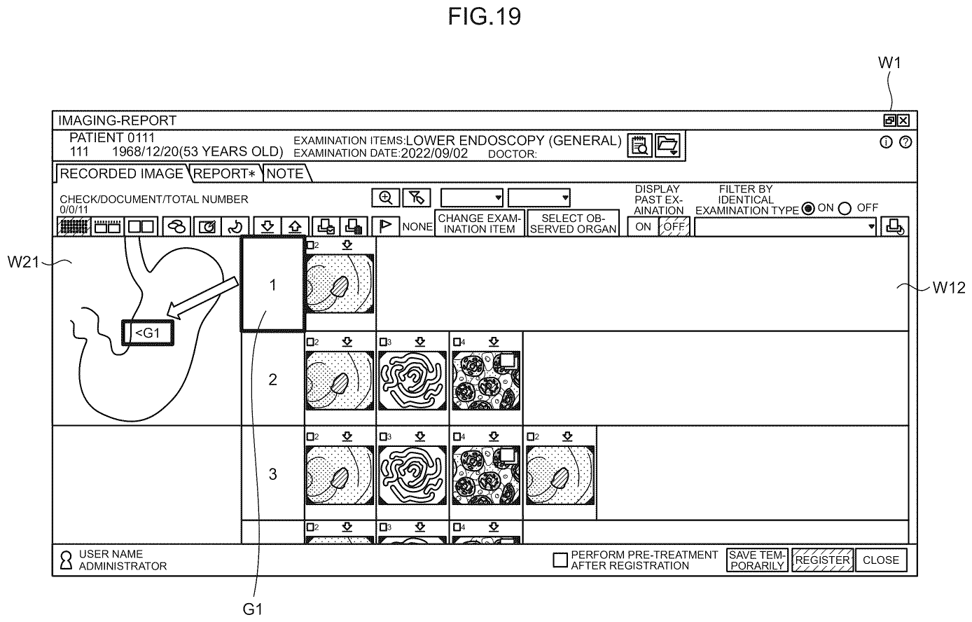

FIG. 19 is a diagram schematically illustrating details of editing performed by the editing unit 666. As illustrated in FIG. 19, the editing unit 666 implements the details of the operation performed by the user on the input unit 63, for example, performs drag operation and drop operation on a field with the group number of G1, and performs editing of associating the group number of “G1” with a predetermined region in the schema diagram W21. After Step S203, the medical support apparatus 6 goes to Step S204 to be described below.

Referring back to FIG. 15, explanation from Step S204 will be continued.

At Step S204, the determining unit 662 determines whether or not the user has performed termination operation on the input unit 63. When the determining unit 662 determines that the user has performed the termination operation on the input unit 63 (Step S204: Yes), the medical support apparatus 6 returns to the main routine in FIG. 5. In contrast, when the determining unit 662 determines that the user has not performed the termination operation on the input unit 63 (Step S204: No), the medical support apparatus 6 returns to Step S201 as described above.

At Step S205, the determining unit 662 determines whether or not the user has performed report generation operation on the input unit 63. When the determining unit 662 determines that the user has performed the report generation operation on the input unit 63 (Step S205: Yes), the medical support apparatus 6 goes to Step S206 to be described later. In contrast, when the determining unit 662 determines that the user has not performed the report generation operation on the input unit 63 (Step S205: No), the medical support apparatus 6 goes to Step S207 to be described later.

At Step S206, the generating unit 667 generates a report in accordance with the details of the operation performed by the user on the input unit 63.

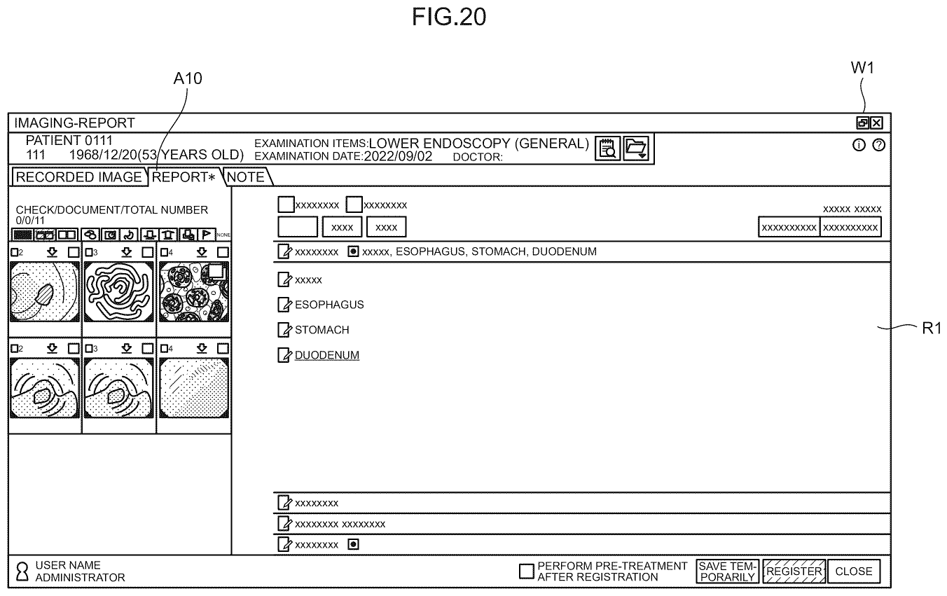

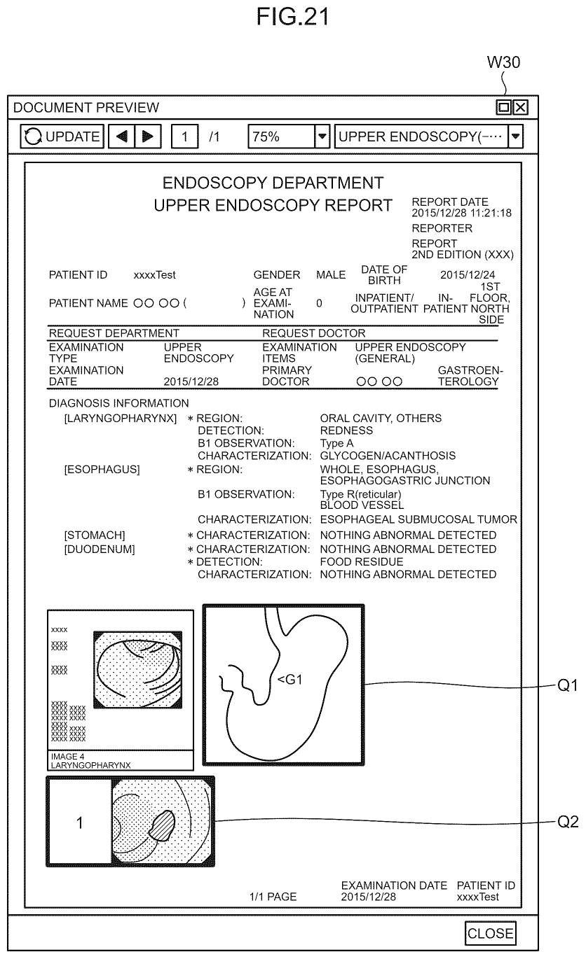

FIG. 20 is a diagram illustrating another example of the imaging-report screen that is displayed by the display unit 62. FIG. 21 is a diagram illustrating an example of the report that is displayed by the display unit 62.

As illustrated in FIG. 20, when the user operates the input unit 63 and selects a report generation sheet A10, the generating unit 667 receives input of diagnosis information on details of diagnosis that is performed by the user on the subject in accordance with details of operation performed by the user, and registers the received diagnosis information. Thereafter, as illustrated in FIG. 21, the generating unit 667 generates a report W30 in which the diagnosis information received in FIG. 20 is reflected. In this case, the display controller 665 displays the report W30 that is generated by the generating unit 667 on the display unit 62. The report W30 includes, in addition to the diagnosis information on the subject, a schema diagram Q1 and a representative image Q2 of the group number that is associated with the schema diagram Q1. With this configuration, the user, by browsing the report W30, is able to recognize a position and an image of a portion subjected to the diagnosis, in addition to the diagnosis information on the diagnosis that is performed by the operator of the endoscope 2. After Step S206, the medical support apparatus 6 goes to Step S204.

Referring back to FIG. 15, explanation from Step S207 will be continued.

At Step S207, the determining unit 662 determines whether or not the user has performed lesion operation on the input unit 63. When the determining unit 662 determines that the user has performed the lesion operation on the input unit 63 (Step S207: Yes), the medical support apparatus 6 goes to Step S208 to be described later. In contrast, when the determining unit 662 determines that the user has not performed the lesion operation on the input unit 63 (Step S207: No), the medical support apparatus 6 goes to Step S209 to be described later.

At Step S208, the display controller 665 performs display and editing for each lesion in accordance with details of operation performed by the user on the input unit 63.

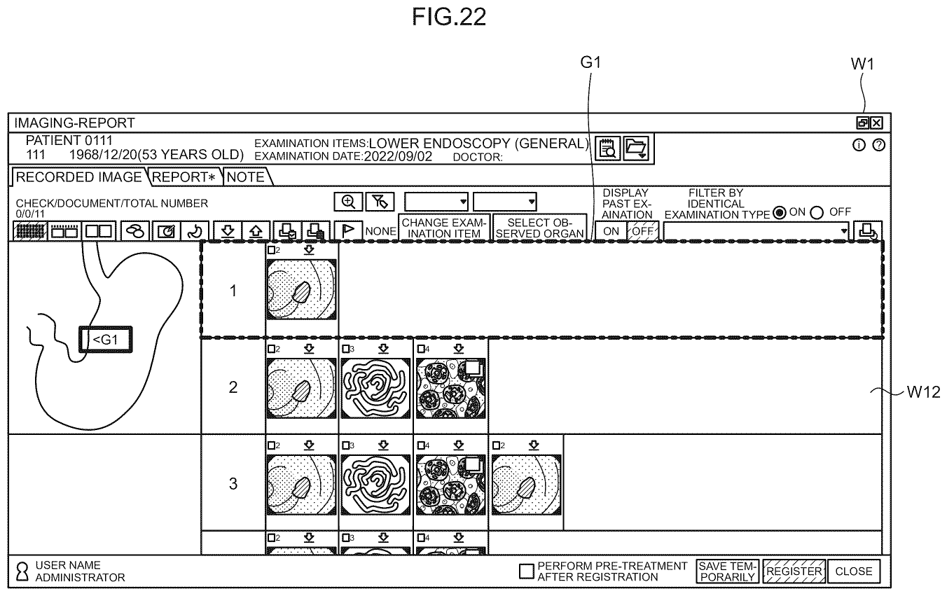

FIG. 22 is a diagram illustrating an example of an image that is displayed by the display unit 62. As illustrated in FIG. 22, when the user operates the input unit 63 and presses the group number of “G1” at a lesion position in the schema diagram W21, the display controller 665 displays an image of the group associated with the group number “G1” in a highlighted manner. With this configuration, the user is able to easily search for the image from the lesion position. After Step S208, the medical support apparatus 6 goes to Step S204.

Referring back to FIG. 15, explanation from Step S209 will be continued.

At Step S209, the determining unit 662 determines whether or not the user has performed past comparison operation on the input unit 63. When the determining unit 662 determines that the user has performed the past comparison operation on the input unit 63 (Step S209: Yes), the medical support apparatus 6 goes to Step S210 to be described later. In contrast, when the determining unit 662 determines that the user has not performed the past comparison operation on the input unit 63 (Step S207: No), the medical support apparatus 6 goes to S204.

At Step S210, the display controller 665 displays a past image in accordance with details of operation performed by the user on the input unit 63.

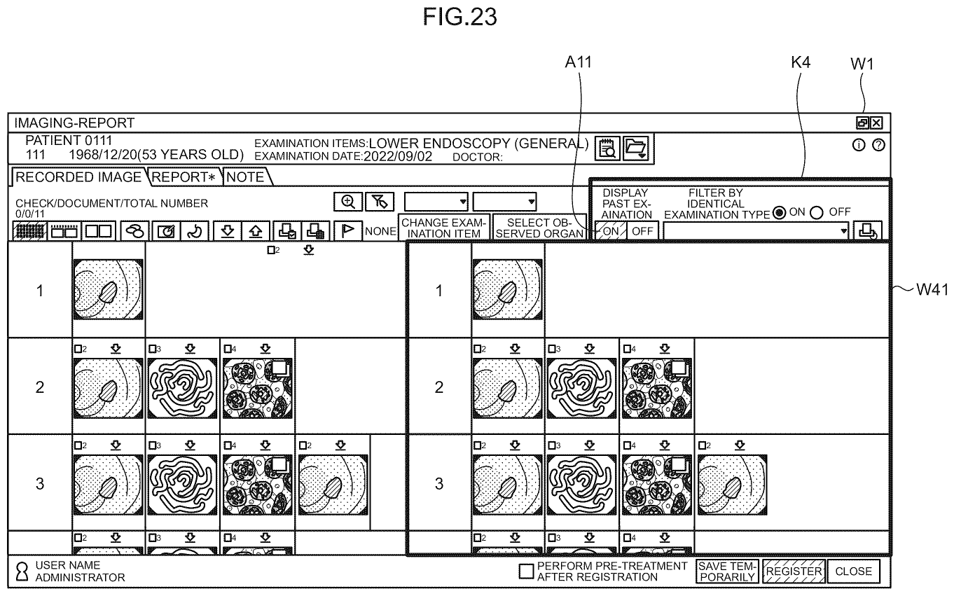

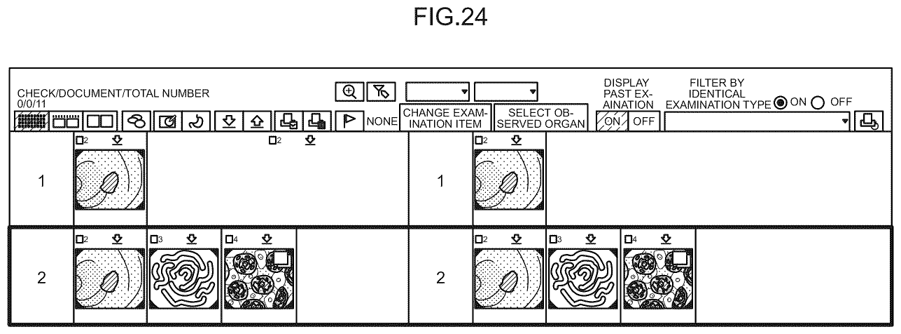

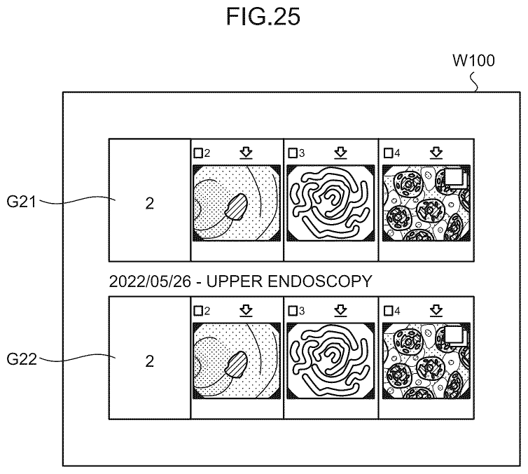

FIG. 23 is a diagram illustrating an example of an image that is displayed by the display unit 62. FIG. 24 is a diagram illustrating an example of the image that is displayed by the display unit 62. FIG. 25 is a diagram illustrating an example of that image that is displayed by the display unit 62.

As illustrated in FIG. 23, when the user operates the input unit 63 and selects an ON icon A11 that designates past examination display in the cell region K4 for receiving input of an instruction signal for displaying a past group image of the same subject, the display controller 665 displays, on the display unit 62, an image group W41 for each group in the examination that has been performed on the subject in the past. Furthermore, when the user operates the input unit 63 and selects the group number to be compared, for example, when the group number of “2” is selected as illustrated in FIG. 24, the display controller 665 displays, as illustrated in FIG. 25, an image W100 in which a current image group G21 and a past image group G22 with the group number of “2” are arranged vertically or horizontally on the display unit 62. With this configuration, the user is able to browse the past image in a comparison manner. After Step S210, the medical support apparatus 6 goes to Step S204.

### Outline of Transmission Process

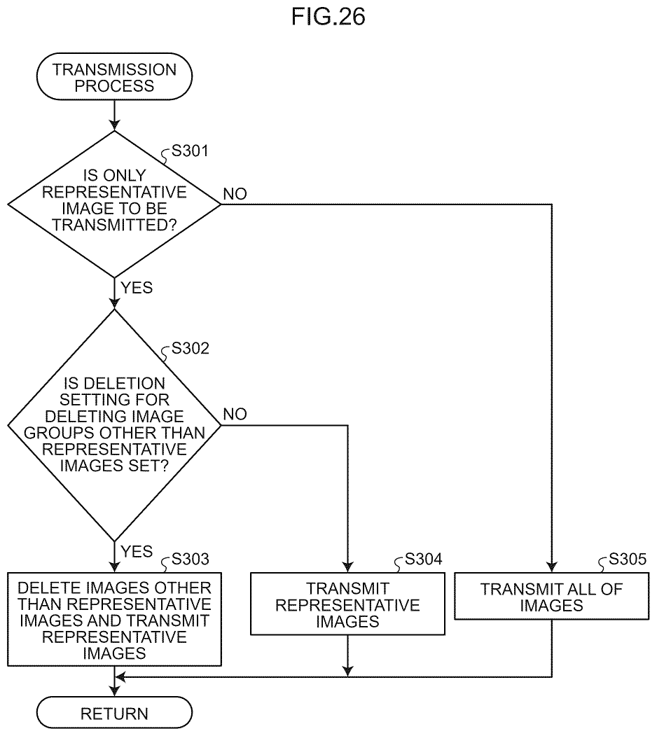

Details of the transmission process described at Step S111 in FIG. 5 will be described in detail below. FIG. 26 is a flowchart illustrating an outline of the transmission process.

As illustrated in FIG. 26, the determining unit 662 determines whether or not to transmit only the representative image of each of the groups (Step S301). Specifically, the determining unit 662 determines whether or not the user has set, in advance, setting information for transmitting only the representative image of each of the groups in the transmission setting information DB 648, and when the setting information for setting only the representative image is set, the determining unit 662 determines that only the representative image of each of the groups is to be transmitted. When the determining unit 662 determines that only the representative image of each of the groups is to be transmitted (Step S301: Yes), the medical support apparatus 6 goes to Step S302 to be described later. In contrast, when the determining unit 662 determines that only the representative image of each of the groups is not to be transmitted (Step S301: No), the medical support apparatus 6 goes to Step S305 to be described later.

At Step S302, the determining unit 662 determines whether or not deletion setting for deleting image groups other than the representative image of each of the groups is set. Specifically, the determining unit 662 determines whether or not the user has set, in advance, the deletion setting for deleting images other than the representative image of each of the groups after transmission of the representative image of each of the groups to the transmission setting information DB 648 is set, and when the deletion setting is set, the determining unit 662 determines that only the representative image of each of the groups is to be transmitted. When the determining unit 662 determines that the deletion setting for deleting image groups other than the representative image of each of the groups is set (Step S302: Yes), the medical support apparatus 6 goes to Step S303 to be described later. In contrast, when the determining unit 662 determines that the deletion setting for deleting image groups other than the representative image of each of the groups is not set (Step S302: No), the medical support apparatus 6 goes to Step S304 to be described later.

At Step S303, the transmission controller 668 deletes images other than the representative image of each of the groups from the DB 64, and transmits only the representative image of each of the groups to the image management server 8. With this configuration, it is possible to reduce an amount of transmission data, and it is possible to increase a storage capacity of the DB 64. After Step S303, the medical support apparatus 6 returns to the main routine in FIG. 5.

At Step S304, the transmission controller 668 transmits only the representative image of each of the groups to the image management server 8. After Step S304, the medical support apparatus 6 returns to the main routine in FIG. 5.

At Step S305, the transmission controller 668 transmits all of the images including the representative image of each of the groups and still image groups to the image management server 8. After Step S305, the medical support apparatus 6 returns to the main routine in FIG. 5.

According to one embodiment as described above, the imaging-report screen that includes the representative image of each of the groups selected by the selector 664 is displayed on the display unit 62, so that it is possible to allow the user to easily and efficiently check an image from a large amount of endoscopic image groups with different magnification.

Furthermore, according to one embodiment, the determining unit 662 determines a plurality of images that include identical feature areas (identical lesions) and classifies the images that include the identical feature areas (identical lesions) as an identical group, so that it is possible to allow the user to easily and efficiently perform checking from among a large amount of endoscopic image groups with different magnification.

Moreover, according to one embodiment, the determining unit 662 adopts a time of an image for which imaging magnification is one-fold magnification that indicates a non-enlarged image among a plurality of images as a start point, adopts a time of an image for which imaging magnification is one-fold magnification that indicates a non-enlarged image among the plurality of images as an end point, and determines a plurality of enlarged images (imaging magnification of two or more) in the adopted period as images with identical feature areas, so that it is possible to allow the user to easily and efficiently perform checking from among a large amount of endoscopic image groups with different magnification.

Furthermore, according to one embodiment, the determining unit 662 determines images that are temporally consecutive non-enlarged images with one-fold magnification, that are captured within a predetermined period (for example, within one minute), and that have identical imaging regions and identical lesion sizes as images that include identical feature areas, so that it is possible to prevent double-check of images by the user.

Moreover, according to one embodiment, the transmission controller 668 deletes images other than the representative image of each of the groups from the DB 64, and transmits only the representative image of each of the groups to the image management server 8, so that it is possible to reduce an amount of transmission data, and it is possible to increase the storage capacity of the DB 64.

Furthermore, according to one embodiment, the transmission controller 668 transmits only the representative image of each of the groups to the image management server 8, so that it is possible to reduce the amount of transmission data.

Moreover, according to one embodiment, the display controller 665 displays an image of each of image files based on selection information for selecting any of an organ, a region, an image group, and a feature area, so that it is possible to efficiently display an image that is desired by the user.

Various embodiments may be made by appropriately combining a plurality of components disclosed in the medical support system according to one embodiment of the present disclosure as described above. For example, some components may be deleted from all of the components of the medical support system according to one embodiment of the present disclosure as described above. Furthermore, the components described in the medical support system according to one embodiment of the present disclosure as described above may be combined appropriate.

Moreover, in the medical support system according to one embodiment of the present disclosure, the “unit” described above may be replaced with a “means”, a “circuit”, or the like. For example, the control unit may be replaced with a control means or a control circuit.

Furthermore, the program to be executed by the medical support system according to one embodiment of the present disclosure are provided by being recorded, as file data in an installable format or executable format, on a computer readable recording medium, such as a CD-ROM, a flexible disk (FD), a CD-R, a digital versatile disk (DVD), a USB medium, or a flash memory.

Moreover, the program to be executed by the medical support system according to one embodiment of the present disclosure may be stored in a computer that is connected to a network, such as the Internet, and may be provided by being downloaded via the network.

Meanwhile, in the description of the flowcharts in the present specification, the context of processes between steps is clearly indicated using expressions such as “first”, “after that”, “subsequently”, etc. However, the order of the processes required for implementing the disclosure is not uniquely determined by these expressions. That is, the order of processes in the flowcharts described in this specification may be changed within a consistent range. Furthermore, the disclosure is not limited to such a program including a simple branching process, and a larger number of items may be comprehensively determined to be branched.

According to the present disclosure, it is possible to easily and efficiently check a desired image from a large amount of endoscopic image groups with different magnification.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical support apparatus comprising:
a processor comprising hardware, the processor being configured to
acquire a plurality of image files in which a plurality of images of an inside body of a subject captured in temporal sequence at multiple imaging magnifications, imaging magnification information on the imaging magnifications, and imaging times of the plurality of images are associated,
based on the imaging magnification information, set an image having a first imaging magnification among the plurality of images to a first image, set an image having a next first imaging magnification on in time series among the plurality of images to a second image, and determine images captured in a period from an imaging time of the first image to an imaging time of the second image as third images that include identical feature areas,
group, based on a result of the determination result, the third images into an image group,
select a representative image of the image group, and
display the representative image on a display.

2. The medical support apparatus according to claim 1, wherein
the processor is further configured to
acquire size information that indicates sizes of the feature areas and region information on either an organ or a region in the inside body of the subject at a time of capturing the plurality of images, and
determine, as the third images, fourth images for which a detection time of the region information and a detection time of the size information fall within a predetermined period among the plurality of images, the fourth images having a second imaging magnification.

3. The medical support apparatus according to claim 2, wherein
the processor is further configured to
acquire treatment information on treatment of a treatment tool, and
determine, as the third images, fifth images which are obtained after an image having a maximum imaging magnification is obtained among the plurality of images, which have the first imaging magnification, and for which the detection time of the region information, the detection time of the size information, and a detection time of the treatment information fall within a predetermined period.

4. The medical support apparatus according to claim 3, wherein the processor is configured to transmit only the representative image of the image group when transmitting the plurality of image files to an image management server managing images.

5. The medical support apparatus according to claim 4, wherein the processor is configured to delete the plurality of image files other than the representative image transmitted to the image management server.

6. The medical support apparatus according to claim 5, wherein after transmission of the representative image to the image management server, the processor is configured to delete the plurality of image files other than the representative image after a lapse of a predetermined time.

7. The medical support apparatus according to claim 6, wherein the processor is configured to display, based on selection information for selecting one of an organ, a region, the image group, and the feature areas, the plurality of images of the plurality of image files.

8. The medical support apparatus according to claim 1, wherein
the processor is further configured to
acquire size information that indicates sizes of the feature areas and region information on either an organ or a region in the inside body of the subject at a time of capturing the plurality of images, and
determine, as the third images, sixth images that are included in the plurality of images, have a second imaging magnification, are captured within a predetermined period, have identical sizes of the identical feature areas, and identical organs or regions.

9. The medical support apparatus according to claim 1, wherein
the processor is further configured to
acquire region information on either an organ or a region in the inside body of the subject at a time of capturing the plurality of images and treatment information on treatment of a treatment tool, and
determine, as the third images, a seventh image having a maximum imaging magnification and an eighth image that is obtained after the seventh image, the seventh image and the eighth image being included in the plurality of images, being captured within a predetermined period, having identical organs or regions, and being images in which the treatment is performed within the predetermined period.

10. A medical support system comprising:
an endoscope configured to capture an inside body of a subject;
a controller configured to record a plurality of image files in which a plurality of images of the inside body of the subject captured in temporal sequence at multiple imaging magnifications by the endoscope, imaging magnification information on the imaging magnifications, and imaging times of the plurality of images are associated; and
a medical support apparatus comprising a processor comprising hardware, the processor being configured to
acquire the plurality of image files from the controller,
based on the imaging magnification information, set an image having a first imaging magnification among the plurality of images to a first image, set an image having a next first imaging magnification on in time series among the plurality of images to a second image, and determine images captured in a period from an imaging time of the first image to an imaging time of the second image as third images that include identical feature areas,
group, based on a result of the determination, the third images into an image group,
select a representative image of the image group, and
display the representative image on a display.

11. The medical support system according to claim 10, further comprising
an image diagnosis apparatus configured to detect, as detection result information, a feature area in the inside body of the subject in cooperation with imaging by the endoscope when the image is captured, wherein,
the detection result information includes size information that indicates sizes of the feature areas and region information on either an organ or a region in the inside body of the subject at a time of capturing the plurality of images, and
the processor is configured to determine, as the third images, fourth images for which a detection time of the region information and a detection time of the size information fall within a predetermined period among the plurality of images, the fourth images having a second imaging magnification.

12. The medical support system according to claim 11, wherein
the detection result information further includes treatment information on treatment of a treatment tool, and
the processor is configured to determines, as the third images, fifth images which are obtained after an image having maximum imaging magnification is obtained among the plurality of images, which have the first imaging magnification, and for which the detection time of the region information, the detection time of the size information, and a detection time of the treatment information fall within a predetermined period.

13. A medical support method comprising
acquiring a plurality of image files in which a plurality of images of an inside body of a subject captured in temporal sequence at multiple imaging magnifications, imaging magnification information on the imaging magnifications, and an imaging times of the plurality of images are associated,
based on the imaging magnification information, setting an image having a first imaging magnification among the plurality of images to a first image, setting an image having a next first imaging magnification on in time series among the plurality of images to a second image, and determining images captured in a period from an imaging time of the first image to an imaging time of the second image as third images that include identical feature areas,
grouping, based on a result of the determination, the third images into an image group,
selecting a representative image of the image group, and
displaying the representative image on a display.

14. The medical support method according to claim 13, further comprising
acquiring size information that indicates sizes of the feature areas and region information on either an organ or a region in the inside body of the subject at a time of capturing the plurality of images, and
determining, as the third images, fourth images for which a detection time of the region information and a detection time of the size information fall within a predetermined period among the plurality of images, the fourth images having a second imaging magnification.

15. The medical support method according to claim 14, further comprising
acquiring treatment information on treatment of a treatment tool, and
determining, as the third images, fifth images which are obtained after an image having a maximum imaging magnification is obtained among the plurality of images, which have the first imaging magnification, and for which the detection time of the region information, the detection time of the size information, and a detection time of the treatment information fall within a predetermined period.

16. The medical support method according to claim 15, further comprising transmitting only the representative image of the image group when transmitting the plurality of image files to an image management server managing images.

17. The medical support method according to claim 16, further comprising deleting the plurality of image files other than the representative image transmitted to the image management server.

18. The medical support method according to claim 17, further comprising
after transmission of the representative image to the image management server, deleting the plurality of image files other than the representative image after a lapse of a predetermined time.

19. The medical support method according to claim 18, further comprising
display, based on selection information for selecting one of an organ, a region, the image group, and the feature area, the plurality of images of the plurality of image files.
